(19) United States
(12) Patent Application Publication
(10) Pub. No.: US 2025/0304712 A1
(43) Pub. Date: Oct. 2, 2025

(54) ANTIBODY SEQUENCES OF THREE ANTIBODY CANDIDATES TARGETING HUMAN LYMPHOCYTE ANTIGEN 6 FAMILY MEMBER K (LY6K)

(71) Applicants: National University of Singapore, Singapore (SG); Yissum Research Development Company of the Hebrew University of Jerusalem Ltd, Jerusalem (IL); Hadasit Medical Research Services And Development Ltd, Jerusalem (IL)

(72) Inventors: Paul Anthony Macary, Singapore (SG); Manuel Adrian Suter, Singapore (SG); Si Min Lang, Singapore (SG); Yoav Moshe Smith, Jerusalem (IL); Hovav Nechushtan, Jerusalem (IL)

(73) Assignees: National University of Singapore, Singapore (SG); Yissum Research Development Company of the Hebrew University of Jerusalem Ltd, Jerusalem (IL); Hadasit Medical Research Services And Development Ltd, Jerusalem (IL)

(21) Appl. No.: 18/728,793

(22) PCT Filed: Jan. 11, 2023
(86) PCT No.: PCT/SG2023/050022

§ 371 Date: Jul. 12, 2024

(30) Foreign Application Priority Data
Jan. 14, 2022 (SG) 10202200405Q

(51) Int. Cl. C07K 16/30; A61K 47/68; A61P 35/00; G01N 33/569; G01N 33/574

(52) U.S. Cl. CPC C07K 16/30; A61K 47/6851; A61P 35/00; G01N 33/56966; G01N 33/574; C07K 2317/24; C07K 2317/565; C07K 2317/732; C07K 2317/77; G01N 2333/47

(57) ABSTRACT

The present invention relates to isolated antibodies, fragments thereof and antibody-drug conjugates that bind to Lymphocyte antigen 6 family member K (LY6K) and their use for screening for LY6K-positive malignant cells or for therapeutic targeting of LY6K-positive malignant cells.

### FIELD OF THE INVENTION

The present invention relates to isolated antibodies, fragments thereof and antibody-drug conjugates that bind to Lymphocyte antigen 6 family member K (LY6K) and their use for screening for LY6K-positive malignant cells or for therapeutic targeting of LY6K-positive malignant cells.

### BACKGROUND

Antibody-based therapies have shown enormous clinical benefits in patients suffering from certain types of hematologic and solid malignancies. One of the biggest challenges in antibody therapies, however, remains the identification of target antigens that are upregulated in a cancer-specific manner with minimal or no expression in healthy tissues. Cancer/testis antigens (CTAs) are often aberrantly expressed in cancer, and with the exception of testis and germline-derived cells, are largely absent in healthy tissues. Thus, CTAs constitute promising target antigens for immunotherapies against cancer. However, developing antibody-based strategies has been complicated by the fact that most CTAs are expressed intracellularly.

There is a need to provide improved antibodies.

### SUMMARY OF THE INVENTION

To address the above issues, the inventors employed a bioinformatics approach to identify CTAs with putative cell surface expression. The inventors identified several CTAs, of which Lymphocyte antigen 6 family member K (LY6K) was the most consistently upregulated target antigen in cervical, tongue, lung adenocarcinoma as well as lung squamous cell carcinoma.

According to a first aspect, the present invention provides an isolated antibody or a fragment thereof that specifically binds to at least one epitope of Lymphocyte antigen 6 family member K (LY6K), wherein the antibody or fragment thereof comprises three complementarity determining region sequences (CDR1, CDR2 and CDR3) in at least one variable light chain and three complementarity determining region sequences (CDR1, CDR2 and CDR3) in at least one variable heavy chain, wherein:
a) the light chain comprises an amino acid sequence having at least 90% sequence identity to a light chain selected from the group comprising SEQ ID NO: 7 and SEQ ID NO: 31, and the variable heavy chain comprises an amino acid sequence having at least 90% sequence identity to a light chain selected from the group comprising SEQ ID NO: 8 and SEQ ID NO: 32; or
b) the light chain comprises an amino acid sequence having at least 90% sequence identity to a light chain selected from the group comprising SEQ ID NO: 17, SEQ ID NO: 35, SEQ ID NO: 36 and SEQ ID NO: 37, and the variable heavy chain comprises an amino acid sequence having at least 90% sequence identity to a light chain selected from the group comprising SEQ ID NO: 18, SEQ ID NO: 38 and SEQ ID NO: 39; or
c) the light chain comprises an amino acid sequence having at least 90% sequence identity to a light chain selected from the group comprising SEQ ID NO: 27, SEQ ID NO: 45 and SEQ ID NO: 46, and the variable heavy chain comprises an amino acid sequence having at least 90% sequence identity to a light chain selected from the group comprising SEQ ID NO: 28, SEQ ID NO: 47 and SEQ ID NO: 48.

Determination of CDR sequences can be made according to any method known in the art, including but not limited to the methods known as KABAT, Chothia and IMGT. A selected set of CDRs may include sequences identified by more than one method, namely, some CDR sequences may be determined using KABAT and some using IMGT, for example.

In some embodiments:
a) the variable light chain amino acid sequence comprises six complementarity determining regions (CDRs), wherein the light chain CDR1 comprises the amino acid sequence KSSQSLLASDGKTYLN set forth in SEQ ID NO: 1; the light chain CDR2 comprises the amino acid sequence LVSKLDS set forth in SEQ ID NO: 2; the light chain CDR3 comprises the amino acid sequence WQGSHFPLT set forth in SEQ ID NO: 3; the heavy chain CDR1 comprises the amino acid sequence SYWMH set forth in SEQ ID NO: 4; the heavy chain CDR2 comprises the amino acid sequence EINPSNGGANYNEKFKR set forth in SEQ ID NO: 5; and the heavy chain CDR3 comprises the amino acid sequence GGNFFFAY set forth in SEQ ID NO: 6; or
b) the variable light chain amino acid sequence comprises six complementarity determining regions (CDRs), wherein the light chain CDR1 comprises the amino acid sequence RSSQSLVHTDGDTYLN set forth in SEQ ID NO: 11; the light chain CDR2 comprises the amino acid sequence KVSNRFS set forth in SEQ ID NO: 12; the light chain CDR3 comprises the amino acid sequence SQNTHVYT set forth in SEQ ID NO: 13; the heavy chain CDR1 comprises the amino acid sequence SYGMS set forth in SEQ ID NO: 14; the heavy chain CDR2 comprises the amino acid sequence TISSGGTYTYYSDSVKG set forth in SEQ ID NO: 15; and the heavy chain CDR3 comprises the amino acid sequence HDWDFFDY set forth in SEQ ID NO: 16; or
c) the variable light chain amino acid sequence comprises six complementarity determining regions (CDRs), wherein the light chain CDR1 comprises the amino acid sequence CRSSQTILHSNGNTYLE set forth in SEQ ID NO: 21; the light chain CDR2 comprises the amino acid sequence KVSNRLS set forth in SEQ ID NO: 22; the light chain CDR3 comprises the amino acid sequence FQGSHDPFT set forth in SEQ ID NO: 23; the heavy chain CDR1 comprises the amino acid sequence NFAMN set forth in SEQ ID NO: 24; the heavy chain CDR2 comprises the amino acid sequence WINTNTGEPTYVEEFKG set forth in SEQ ID NO: 25; and the heavy chain CDR3 comprises the amino acid sequence REWRRSYYFDY set forth in SEQ ID NO: 26.

In some embodiments,
a) the light chain comprises an amino acid sequence having at least 90% sequence identity to a light chain selected from the group comprising SEQ ID NO: 7 and SEQ ID NO: 31, and the variable heavy chain comprises an amino acid sequence having at least 90% sequence identity to a light chain selected from the group comprising SEQ ID NO: 8 and SEQ ID NO: 32; or
b) the light chain comprises an amino acid sequence having at least 90% sequence identity to a light chain selected from the group comprising SEQ ID NO: 17, SEQ ID NO: 35, SEQ ID NO: 36 and SEQ ID NO: 37, and the variable heavy chain comprises an amino acid sequence having at least 90% sequence identity to a light chain selected from the group comprising SEQ ID NO: 18, SEQ ID NO: 38 and SEQ ID NO: 39; or
c) the light chain comprises an amino acid sequence having at least 90% sequence identity to a light chain selected from the group comprising SEQ ID NO: 27, SEQ ID NO: 45 and SEQ ID NO: 46, and the variable heavy chain comprises an amino acid sequence having at least 90% sequence identity to a light chain selected from the group comprising SEQ ID NO: 28, SEQ ID NO: 47 and SEQ ID NO: 48.

In some embodiments, the antibody is a monoclonal mouse, humanized or chimeric antibody.

Antibodies of the invention were found to have a level of cytotoxicity in and of themselves. It is known that anti-tumor efficacy of therapeutic antibodies can be achieved through downstream signaling events such as growth and proliferation inhibition initiating apoptosis or by activating the patient's immune system, resulting in complement or antibody dependent cellular cytotoxicity (ADCC) [Chalouni, C., Doll, S. J Exp Clin Cancer Res 37:20 (2018)].

Antibody-drug conjugates (ADCs) are usually composed of a humanized or chimeric antibody chemically linked to a cytotoxic drug allowing the delivery of cytotoxic drug specifically to antigen-positive malignant cells [Chalouni, C., Doll, S. J Exp Clin Cancer Res 37:20 (2018)]. The antibody's specificity and the local release of cytotoxic drug are the main parameters that provide increased anti-tumor efficacy and decreased systemic toxicity. Therefore, ADCs have a wider therapeutic window compared to traditional chemotherapy. A chemical linker is used to attach the cytotoxic drug to the antibody, and the physicochemical properties of the linker largely determine the pharmacokinetics of an ADC. Linkers are usually classified as “cleavable” and “non-cleavable”. They are designed to be highly stable in the circulation to limit systemic toxicity and to be readily cleavable once the ADC reaches its intracellular destination to deliver the payload. “Cleavable” linkers are designed to allow the release of the drug by hydrolysis (low pH, reduction of disulfide bonds) or by proteolysis. Linkers designed for proteolysis contain sites specifically recognized by certain enzymes, such as cysteine proteases. “Non-cleavable” linkers rely on the degradation of the antibody itself to release their cytotoxic payload. There are two main categories of cytotoxic drugs [Chalouni, C., Doll, S. J Exp Clin Cancer Res 37:20 (2018)]: microtubule inhibitors and DNA damaging drugs.

In some embodiments, the said isolated antibody or fragment thereof is an antibody conjugate.

In some embodiments, the said isolated antibody or fragment thereof is conjugated to a cytotoxic drug, a radioactive moiety, or an identifiable moiety such as a fluorescent tag or biotin.

According to a second aspect, the present invention provides a pharmaceutical composition comprising the isolated antibody, fragment or drug-conjugate thereof defined in aspect 1 and a pharmaceutical acceptable excipient, diluent, salt, or carrier.

According to a third aspect, the present invention provides an isolated antibody or fragment thereof according to aspect 1, or a pharmaceutical composition according to aspect 2, for use in the treatment of LY6K-positive malignant cells.

In some embodiments, the LY6K-positive malignant cells are selected from the group comprising cervical cancer, lung adenocarcinoma, lung squamous cell carcinoma (SCC), tongue SCC, head and neck, and breast cancer.

According to a fourth aspect, the present invention provides use of at least one antibody or fragment thereof defined in aspect 1 for the preparation of a medicament for treatment of a LY6K-positive malignant cell disease.

In some embodiments, the LY6K-positive malignant cell disease is selected from the group consisting of cancer, such as cervical cancer, lung adenocarcinoma, lung squamous cell carcinoma (SCC), tongue SCC, head and neck, and breast cancer.

According to a fifth aspect, the present invention provides a method of treating a LY6K-positive malignant cell disease, the method comprising administering to a subject an antibody or fragment thereof defined in aspect 1.

In some embodiments, the LY6K-positive malignant cell disease is selected from the group consisting of cancer, such as cervical cancer, lung adenocarcinoma, lung squamous cell carcinoma (SCC), tongue SCC, head and neck, and breast cancer.

In some embodiments, the method further comprises administering an additional anti-cancer therapy selected from surgery, chemotherapy, radiotherapy, and immunotherapy.

According to a sixth aspect, the present invention provides a method of detecting a LY6K-positive malignant cell in a biological sample, comprising:
i) providing at least one biological sample;
ii) contacting the at least one biological sample with an antibody or fragment thereof of aspect 1;
iii) detecting specific binding; and
iv) comparing the binding in iii) with a control.

The present invention also provides polynucleic acids that encode the variable light and heavy domains of the antibodies of the invention.

According to an eighth aspect, the present invention provides an isolated nucleic acid molecule encoding
(a) at least one variable light chain of the antibody defined in claim 2 or 5, wherein the variable light chain comprises at least one amino acid sequence selected from the group consisting of SEQ ID NO: 7 and SEQ ID NO: 31, and (b) at least one variable heavy chain of the antibody defined in claim 1 or 5, wherein the variable heavy chain comprises at least one amino acid sequence selected from the group consisting of SEQ ID NO: 8 and SEQ ID NO: 32; or
(c) at least one variable light chain of the antibody defined in claim 2 or 5, wherein the variable light chain comprises at least one amino acid sequence selected from the group consisting of SEQ ID NO: 17, SEQ ID NO: 35, SEQ ID NO: 36 and SEQ ID NO: 37, and (d) at least one variable heavy chain of the antibody defined in claim 1 or 5, wherein the variable heavy chain comprises at least one amino acid sequence selected from the group consisting of SEQ ID NO: 18, SEQ ID NO: 38 and SEQ ID NO: 39; or
(e) at least one variable light chain of the antibody defined in claim 2 or 5, wherein the variable light chain comprises at least one amino acid sequence selected from the group consisting of SEQ ID NO: 27, SEQ ID NO: 45 and SEQ ID NO: 46, and (f) at least one variable heavy chain of the antibody defined in claim 1 or 5, wherein the variable heavy chain comprises at least one amino acid sequence selected from the group consisting of SEQ ID NO: 28, SEQ ID NO: 47 and SEQ ID NO: 48.

In some embodiments,
i) the at least one nucleic acid sequence in (a) has at least 80%, at least 90% or at least 95% sequence identity to SEQ ID NO: 9 and/or SEQ ID NO: 33; and the at least one nucleic acid sequence in (b) has at least 80%, at least 90% or at least 95% sequence identity to SEQ ID NO: 10 and/or SEQ ID NO: 34; or
ii) the nucleic acid sequence in (c) has at least 80%, at least 90% or at least 95% sequence identity to SEQ ID NO: 19, SEQ ID NO: 40, SEQ ID NO: 41 and/or SEQ ID NO: 42; and the at least one nucleic acid sequence in (d) has at least 80%, at least 90% or at least 95% sequence identity to SEQ ID NO: 20, SEQ ID NO: 43 and/or SEQ ID NO: 44; or
iii) the nucleic acid sequence in (e) has at least 80%, at least 90% or at least 95% sequence identity to SEQ ID NO: 29, SEQ ID NO: 49 and/or SEQ ID NO: 50; and the at least one nucleic acid sequence in (f) has at least 80%, at least 90% or at least 95% sequence identity to SEQ ID NO: 30, SEQ ID NO: 51 and/or SEQ ID NO: 52.

According to a ninth aspect, the present invention provides an expression vector comprising at least one of isolated nucleic acid molecule defined in aspect 8.

According to a tenth aspect, the present invention provides a host cell comprising the expression vector defined in aspect 9.

According to an eleventh aspect, the present invention provides a kit comprising at least one antibody or fragment thereof defined in aspect 1.

It will be appreciated that the present invention is not limited to the specific embodiments described in detail below.

### BRIEF DESCRIPTION OF THE FIGURES

FIG. 1A-C shows LY6K cell surface expression. (A) LY6K is expressed on the cell surface of human cancer cell lines. Assessment of LY6K cell surface expression was performed by flow cytometry. Representative histograms are shown for two breast (MDA-MB-231 and MCF7), one lung (Calu-1) and one liver (HUH7) cancer cell line. LY6K (black line) was detected on the cell surface of breast and lung cells, but not on liver cells. Isotype antibody was used as control (filled histogram). (B) Quantification of LY6K expression levels on the cell surface of human cancer cell lines. LY6K was detected at high levels on the cell surface of cervical, head & neck, lung and some breast cancer cell lines. Low levels were found on hematologic, colorectal, liver and pancreatic cell lines. Tissue of origin for cancer cell lines is indicated. Data represent mean±s.e.m. of three independent experiments. (C) Detection of LY6K on the cell surface through confocal microscopy. The plasma membrane of MDA-MB-231, MCF7 (both breast), Calu-1 (lung) and HUH7 (liver) cells were stained using red membrane dye (CellMask) and green anti-LY6K antibody. Scale bar denotes 20 μm.

FIG. 2 shows that expressed monoclonal anti-LY6K antibodies bind to recombinant LY6K. Hybridoma-derived heavy and light chains were cloned into the expression vectors pTT5LnX-MoG1v2 and pTT5LnX-Kappa, respectively. Expressed antibodies were run on SDS-PAGE gel to check for purify and tested on ELISA using recombinant LY6K protein. Since recombinant LY6K harboured a histidine as well as a Strep tag, the inventors included His and Strep as control, to ascertain that the antibodies do not bind either tag.

FIG. 3 shows the assessment of real-time binding characteristics of antibodies to recombinant LY6K. Representative sensograms showing various concentrations of antibodies binding to immobilized recombinant LY6K. Equilibrium dissociation constant (KD) indicates that all antibody candidates have high binding affinity towards LY6K in the low nanomolar range. Highest number of binding sites (Bₘₐₓ) was observed for antibody 28C1, followed by antibody G57 and antibody 22C-G8. Standard error indicated in brackets.

FIG. 4 shows that anti-LY6K antibodies bind various human cancer cell lines. Flow cytometry analysis was performed to assess binding of antibody candidates to various human cancer cell lines deriving from different tissues (A): breast carcinoma; (B): cervical carcinoma; (C): lung carcinoma; (D): pancreatic carcinoma). Percentage of cells expressing LY6K over isotype control is shown and respective cell lines are indicated.

FIG. 5 shows that anti-LY6K antibodies 22C-G8 and 28C1 display higher binding to LY6K expressed on the surface of Ca Ski cells compared to four other commercially available antibodies Assessment of binding to LY6K was performed by flow cytometry on cervical cancer cell line, Ca Ski, and representative histograms are as shown. Isotype antibody (left histogram) was used as the control for staining for LY6K antibodies (right histogram). 22C-G8 and 28C1 showed the highest binding to LY6K at 90.4% and 95.6% respectively. This is followed by clone G-11 (Santa Cruz) at 85.8%, clone #750747 (R&D Systems) at 27.7%, clone CL2435 (Sigma Aldrich) at 4.64% and clone CL2433 (Invitrogen) at 0.49%.

FIG. 6 shows that antibody-mediated complement-dependent cytotoxicity of cervical cancer cells. Antibody candidates G57 and 28C1 elicited complement-dependent cytotoxicity (CDC) of cervical cancer cells (Ca-Ski) at concentrations of 10 and 50 μg/ml. In contrast, minor CDC killing was observed for antibody candidate 22C-G8 only at a concentration of 50 μg/ml. Isotype control (ITC) used as negative control.

FIG. 7 shows immunohistochemistry on tongue and lung tissue microarrays using anti-LY6K antibodies. Formalin-fixed, paraffin-embedded tongue squamous cell carcinoma (SCC), lung adenocarcinoma and lung SCC were stained with antibody candidate 28C1 and binding was detected by an HRP-conjugated secondary antibody (dark grey staining). Samples were counterstained using Hematoxylin (grey staining).

FIG. 8 shows the humanization of anti-LY6K antibodies. Humanized versions of anti-LY6K antibodies 22C-G8, G57 and 28C1 were expressed and run on SDS-PAGE gel to check for purity. ELISA binding assay and flow cytometry assay were performed to check for humanized antibody binding capacity towards recombinant LY6K and the cancer cell line Calu-1, respectively.

FIG. 9 shows the binding profile of humanized 22C-G8 and 28C1 to a panel of cancer cell lines. Humanized 22C-G8 (dark grey outline) and 28C1 (grey outline) retained binding to LY6K expressed on the surface of human tumour cell lines deriving from various origins including breast, cervix, lung and head and neck. Assessment of LY6K cell surface expression was performed by flow cytometry staining using Hu22C-G8 and Hu28C1, followed by detection using secondary anti-human AlexaFluor 647 antibody.

FIG. 10 shows the internalisation of humanized 22C-G8 into Ca Ski cells. (A) Presence of red fluorescence in Ca Ski cells indicates internalisation using confocal microscopy. Isotype antibody and humanized 22C-G8 were covalently labelled with pHRodo Deep Red dye to produce conjugates that fluoresces only in acidic late endosomes and lysosome. Conjugated antibodies (10 μg/mL) were incubated with the Ca Ski cells for 18 hours and stained using green membrane dye (CellMask) and Hoechst 33342 before imaging on FV3000 Confocal Microscope. Scale bar denotes 40 μm. (B) Concentration and time-dependent increase in antibody internalisation In Ca Ski cells. Humanised 22CG8 and isotype control were labelled with Incucyte® Human Fabfluor-pH Red Antibody Labeling Reagent and added to Ca Ski cells at varying concentrations. HD phase and red fluorescence images were captured every 1 hour over 50 hours using 20× magnification. The data were normalized to t=0 and to the isotype control. Data represent mean±standard deviation of triplicates.

FIG. 11 shows that humanized 22C-G8 and 28C1 mediates Antibody Dependent Cellular Cytotoxicity (ADCC). (A) Increased killing of HEK293T-Ly6K overexpressing cells in the presence of Hu22C-G8 and Hu28C1 compared to the isotype control for the three different E: T ratios. HEK293T-Ly6K overexpressing cells were seeded on an E-Plate overnight before the respective antibodies were incubated at a concentration of 25 ug/mL with the cells. Subsequently, NK effector cells isolated from PBMCs via immunomagnetic negative selection were added to the wells at three different E: T ratios at 4:1, 8:1 and 16:1. The killing was monitored using the RTCA xCELLigence system over the course of 60 hours. (B) Specific cytotoxicity induced by Hu22C-G8, Hu28C1 and a combination of Hu22C-G8 and Hu28C1. Specific cytotoxicity was calculated by normalizing the respective cytotoxicity curves to the isotype control curve and plotted over time.

### DETAILED DESCRIPTION OF THE INVENTION

Bibliographic references mentioned in the present specification are for convenience listed in the form of a list of references and added at the end of the examples. The whole content of such bibliographic references is herein incorporated by reference.

### Definitions

Certain terms employed in the specification, examples and appended claims are collected here for convenience.

As used herein, the term “comprising” or “including” is to be interpreted as specifying the presence of the stated features, integers, steps or components as referred to, but does not preclude the presence or addition of one or more features, integers, steps or components, or groups thereof. However, in context with the present disclosure, the term “comprising” or “including” also includes “consisting of”. The variations of the word “comprising”, such as “comprise” and “comprises”, and “including”, such as “include” and “includes”, have correspondingly varied meanings.

As used herein, the term “antibody” refers to any immunoglobulin or intact molecule as well as to fragments thereof that bind to a specific epitope. Such antibodies include, but are not limited to, polyclonal, monoclonal, chimeric, humanised, single chain, single chain fragment variable (scFv), Fab, Fab′, F (ab)′ fragments and/or F (v) portions of the whole antibody. The term “monoclonal antibody” may be referred to as “Mab”. The antibody includes antibodies 22C-G8, G57, and 28C1, produced by hybridoma cell lines or recombinantly. The antibodies, 22C-G8, G57, and 28C1 may be monoclonal antibodies, polyclonal antibodies, single-chain antibodies, and fragments thereof which retain the antigen binding function of the parent antibody. The antibodies 22C-G8, G57, and 28C1 are capable of specifically binding to LY6K and include monoclonal antibodies, polyclonal antibodies, single-chain antibodies, and fragments thereof which retain the antigen binding function of the parent antibody.

The term “antibody fragment” as used herein refers to an incomplete or isolated portion of the full sequence of the antibody which retains the antigen binding function of the parent antibody. Examples of antibody fragments include scFv, Fab, Fab′, F(ab′)2, and Fv fragments; diabodies; linear antibodies; single-chain antibody molecules; and multispecific antibodies formed from antibody fragments. Fragments of the 22C-G8, G57, and 28C1 antibodies are encompassed by the invention so long as they retain the desired affinity of the full-length antibody.

The term “humanized antibody,” as used herein, refers to at least one antibody molecule in which the amino acid sequence in the non-antigen binding regions has been altered so that the antibody more closely resembles a human antibody, and still retains its original binding ability. Such antibodies are included in Table 1.

As used herein, the term “hybridoma” refers to cells that have been engineered to produce a desired antibody in large amounts. For example, to produce at least one hybridoma, B cells are removed from the spleen of an animal that has been challenged with the relevant antigen and fused with at least one immortalized cell. This fusion is performed by making the cell membranes more permeable. The fused hybrid cells (called hybridomas), will multiply rapidly and indefinitely and will produce at least one antibody.

The term “isolated” is herein defined as a biological component (such as a nucleic acid, peptide or protein) that has been substantially separated, produced apart from, or purified away from other biological components in the cell of the organism in which the component naturally occurs, i.e., other chromosomal and extrachromosomal DNA and RNA, and proteins. Nucleic acids, peptides and proteins which have been isolated thus include nucleic acids and proteins purified by standard purification methods. The term also embraces nucleic acids, peptides and proteins prepared by recombinant expression in a host cell as well as chemically synthesized nucleic acids.

The term “sample,” as used herein, is used in its broadest sense. A biological sample suspected of containing LY6K may comprise a bodily fluid, an extract from a cell, chromosome, organelle, or membrane isolated from a cell, a cell; genomic DNA, RNA, or cDNA (in solution or bound to a solid support), a tissue, a tissue print and the like. Of more particular interest are samples that may comprise LY6K-positive malignant cells.

The term “subject” is herein defined as vertebrate, particularly mammal, more particularly human. For purposes of research, the subject may particularly be at least one animal model, e.g., a mouse, rat and the like. In particular, for treatment of LY6K-linked diseases, the subject may be a human with LY6K-positive cancer cells.

A composition or combination of the present invention will generally be administered as a pharmaceutical formulation in admixture with a pharmaceutically acceptable adjuvant, diluent or carrier, which may be selected with due regard to the intended route of administration and standard pharmaceutical practice. Such pharmaceutically acceptable carriers may be chemically inert to the active compounds and may have no detrimental side effects or toxicity under the conditions of use. Suitable pharmaceutical formulations may be found in, for example, Remington The Science and Practice of Pharmacy, 19th ed., Mack Printing Company, Easton, Pennsylvania (1995). For parenteral administration, a parenterally acceptable aqueous solution may be employed, which is pyrogen free and has requisite pH, isotonicity, and stability. Suitable solutions will be well known to the skilled person, with numerous methods being described in the literature. A brief review of methods of drug delivery may also be found in e.g., Langer, (Science 249:1527 (1990)).

Otherwise, the preparation of suitable formulations may be achieved routinely by the skilled person using routine techniques and/or in accordance with standard and/or accepted pharmaceutical practice.

The amount of a composition or combination in any pharmaceutical formulation used in accordance with the present invention will depend on various factors, such as the severity of the condition to be treated, the particular patient to be treated, as well as the compound(s) which is/are employed. In some embodiments the BMC-VLP displays an antigenic molecule on its surface and functions as a vaccine. In any event, the amount of a composition or combination in the formulation may be determined routinely by the skilled person.

For example, a solid oral composition such as a tablet or capsule may contain from 1 to 99% (w/w) active ingredient; from 0 to 99% (w/w) diluent or filler; from 0 to 20% (w/w) of a disintegrant; from 0 to 5% (w/w) of a lubricant; from 0 to 5% (w/w) of a flow aid; from 0 to 50% (w/w) of a granulating agent or binder; from 0 to 5% (w/w) of an antioxidant; and from 0 to 5% (w/w) of a pigment. A controlled release tablet may in addition contain from 0 to 90% (w/w) of a release-controlling polymer.

A parenteral formulation (such as a solution or suspension for injection or a solution for infusion) may contain from 1 to 50% (w/w) active ingredient; and from 50% (w/w) to 99% (w/w) of a liquid or semisolid carrier or vehicle (e.g. a solvent such as water); and 0-20% (w/w) of one or more other excipients such as buffering agents, antioxidants, suspension stabilisers, tonicity adjusting agents and preservatives.

However, the dose administered to a mammal, particularly a human, in the context of the present invention should be sufficient to effect a therapeutic response in the mammal over a reasonable timeframe. One skilled in the art will recognize that the selection of the exact dose and composition and the most appropriate delivery regimen will also be influenced by inter alia the pharmacological properties of the formulation, the nature and severity of the condition being treated, and the physical condition and mental acuity of the recipient, as well as the potency of the specific compound, the age, condition, body weight, sex and response of the patient to be treated.

Having now generally described the invention, the same will be more readily understood through reference to the following examples which are provided by way of illustration, and are not intended to be limiting of the present invention.

### EXAMPLES

Standard molecular biology techniques known in the art and not specifically described were generally followed as described in Sambrook and Russel, Molecular Cloning: A Laboratory Manual, Cold Springs Harbor Laboratory, New York (2001).

### Example 1

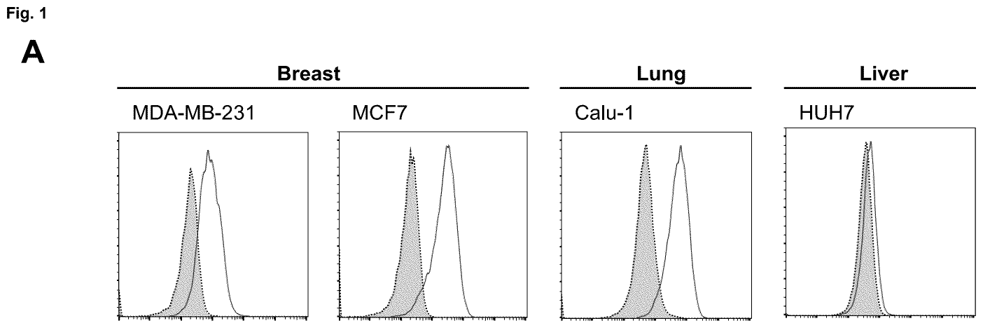

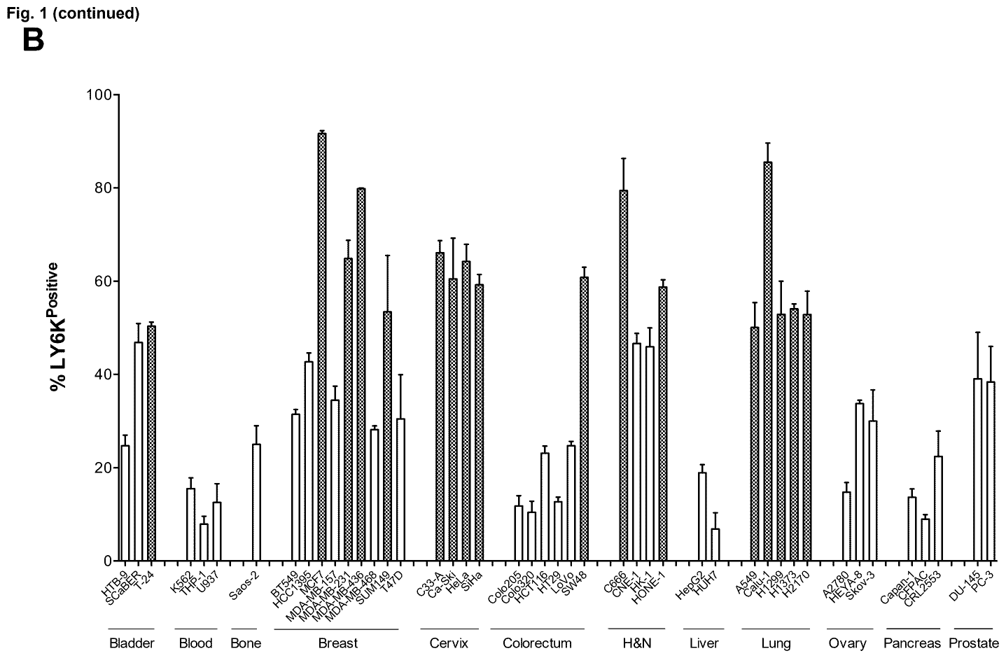

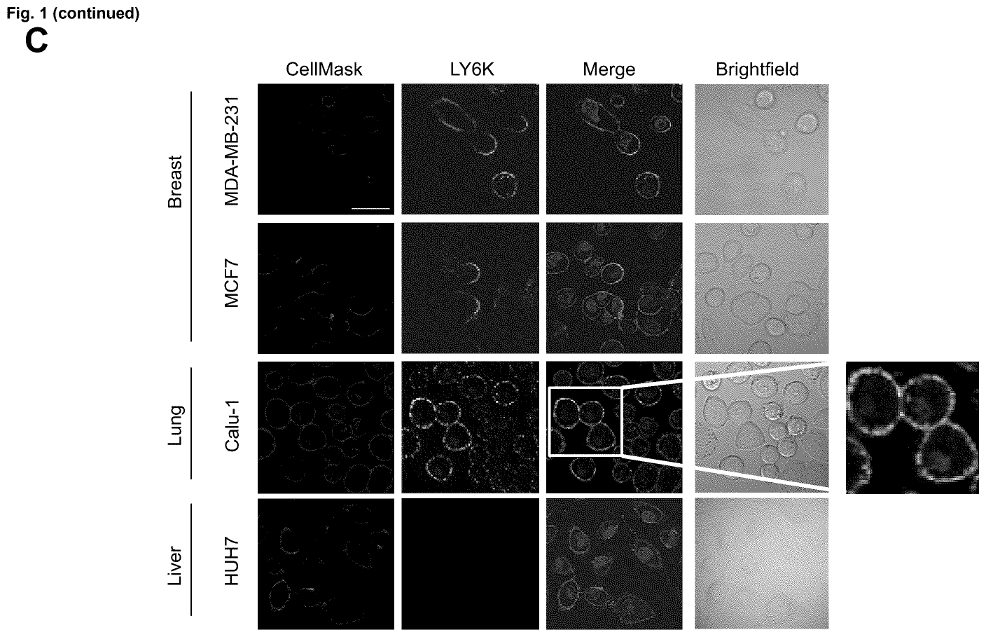

A bioinformatics approach was used to identify CTAs with putative cell surface expression. We identified several CTAs with putative plasma membrane expression, of which LY6K was the most consistently upregulated target antigen in cervical, tongue, lung adenocarcinoma as well as lung squamous cell carcinoma. Given that LY6K is a glycophosphatidylinositol (GPI)-anchored protein, it is conceivable that LY6K is expressed on the cell surface. The inventors assessed the presence of LY6K on the plasma membrane of various human cancer cell lines through flow cytometry analysis and confocal microscopy (FIG. 1). The inventors found that LY6K is present on the cell surface of cervical, head & neck, lung and some of the tested breast cancer cell lines (FIGS. 1A and B). LY6K was not or only minimally expressed on hematological, colorectal, liver and pancreatic cancer cell lines. To corroborate these findings the inventors performed confocal microscopy and stained two breast (MDA-MB-231 and MCF7), one lung (Calu-1) and one liver (HUH7) cancer cell lines using a red cell membrane dye (CellMask) as well as anti-LY6K antibodies (FIG. 1C). LY6K signal clearly overlapped with the cell membrane staining, supporting previous findings that LY6K localizes to the plasma membrane.

### Example 2

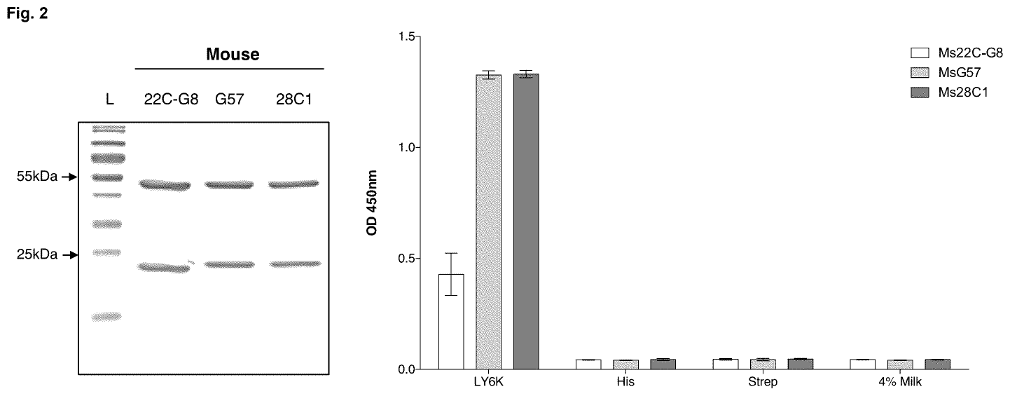

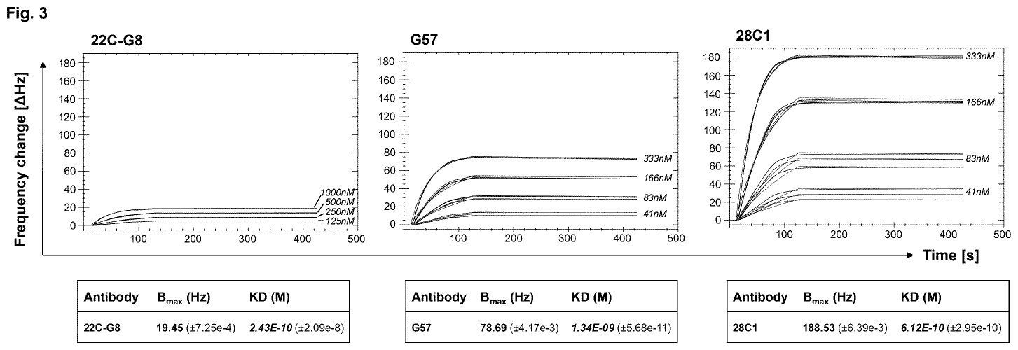

Through employment of hybridoma technology three unique antibodies targeting LY6K (designated 22C-G8, G57 and 28C1) were developed. The inventors cloned the antibody variable fragments of the heavy and light chain into expression vectors. Expressed antibodies were run on an SDS-PAGE gel to check for purity (FIG. 2) and tested binding capacity towards recombinant LY6K through ELISA binding assay (FIG. 2). The inventors next assessed binding affinity towards immobilized LY6K antigen using quartz crystal microbalance (QCM) biosensor. Sensograms for each antibody are shown in FIG. 3 and respective equilibrium dissociation constants (KD) are indicated. KD's for all antibodies are in the low nanomolar range, indicating that all antibodies strongly bind LY6K. Maximum number of binding sites (Bₘₐₓ) is highest for antibody candidate 28C1, suggesting that this antibody binds an epitope that is either more abundant or more easily accessible.

### Example 3

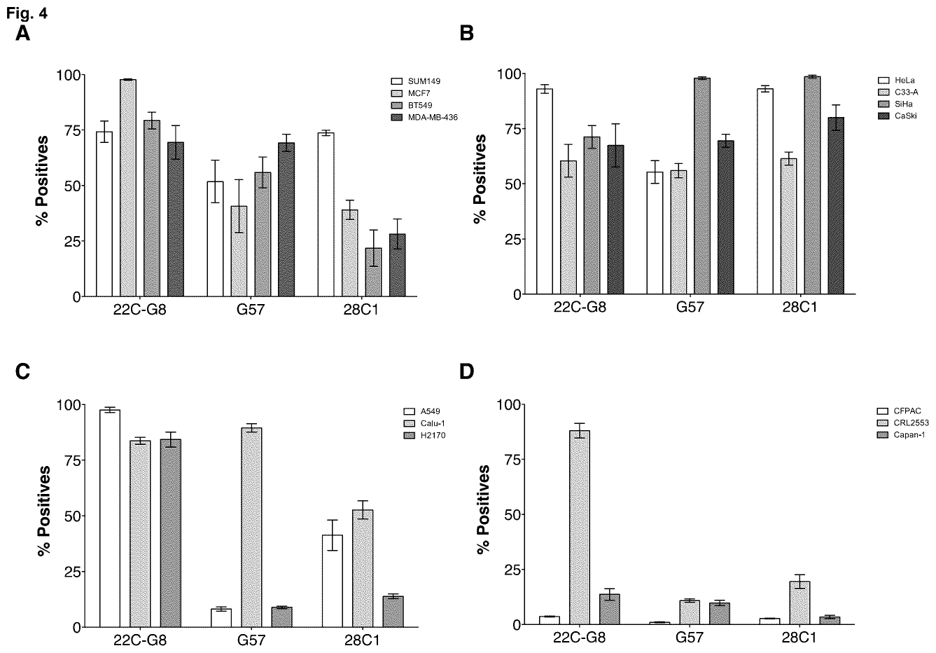

The inventors tested the binding capacity of antibodies 22C-G8, G57 and 28C1 towards human tumour cell lines derived from breast, cervix, lung, and pancreas. 22C-G8 showed high binding to all tested cell lines, whereas G57 bound all cell lines, however to a lesser degree compared to 22C-G8. 28C1 bound strongly only to MDA-MB-157 (FIG. 4A). All antibodies highly bound to all the tested cervical cancer cell lines (FIG. 4B). Binding towards lung cancer cell lines was variable. While 22C-G8 strongly bound to all tested cell lines, 28C1 revealed a much weaker binding profile. The strongest binding was observed for Calu-1 cells. G57 revealed strong binding to Calu-1 but only weak binding to A549 and H2170 (FIG. 4C). Since pancreatic cell lines do not reveal substantial levels of LY6K on their cell surface, those cells were used as negative controls. With the exception of 22CG8, which strongly bound to CRL2553, antibody candidates generally did not bind any of the three tested cell lines (FIG. 4D).

### Example 4

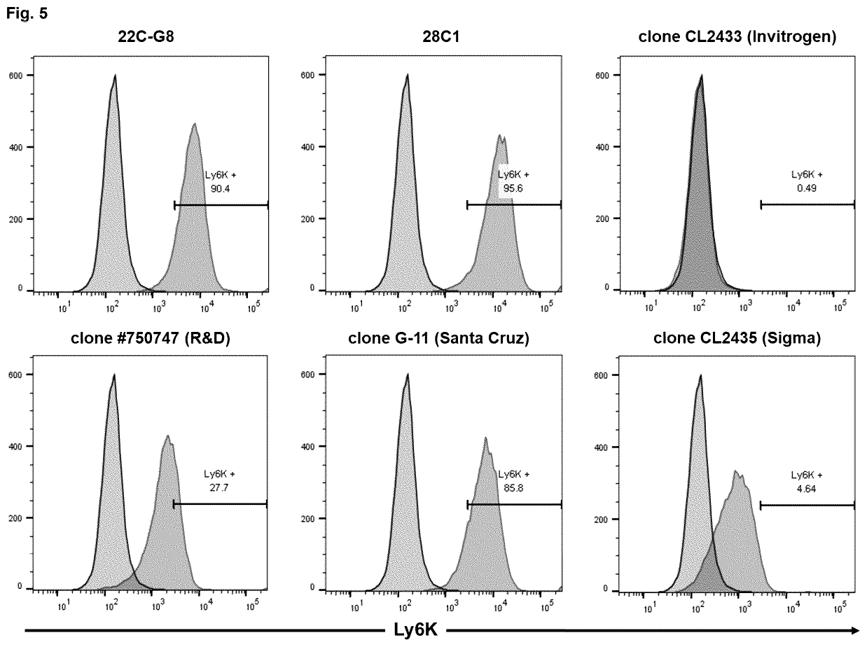

The binding of antibodies 22C-G8 and 28C1 was compared with commercial clones of LY6K antibodies to LY6K expressed on cervical cancer cell line, Ca Ski. Using flow cytometry, the inventors show that the antibodies displayed superior binding to LY6K expressed on the cell surface of Ca Ski cells (FIG. 5). 22C-G8 and 28C1 were among the antibodies that showed the highest binding to LY6K. Clone G-11 (Santa Cruz) also showed comparable binding to LY6K. In comparison, the other antibodies: clone #750747 (R&D Systems), clone CL2435 (Sigma Aldrich) and clone CL2433 (Invitrogen) showed much weaker binding profiles. This result is significant as strong binding to LY6K expressed in its native conformation on the cell surface of cancer cells is a key prerequisite for the development of the antibody into therapeutics.

### Example 5

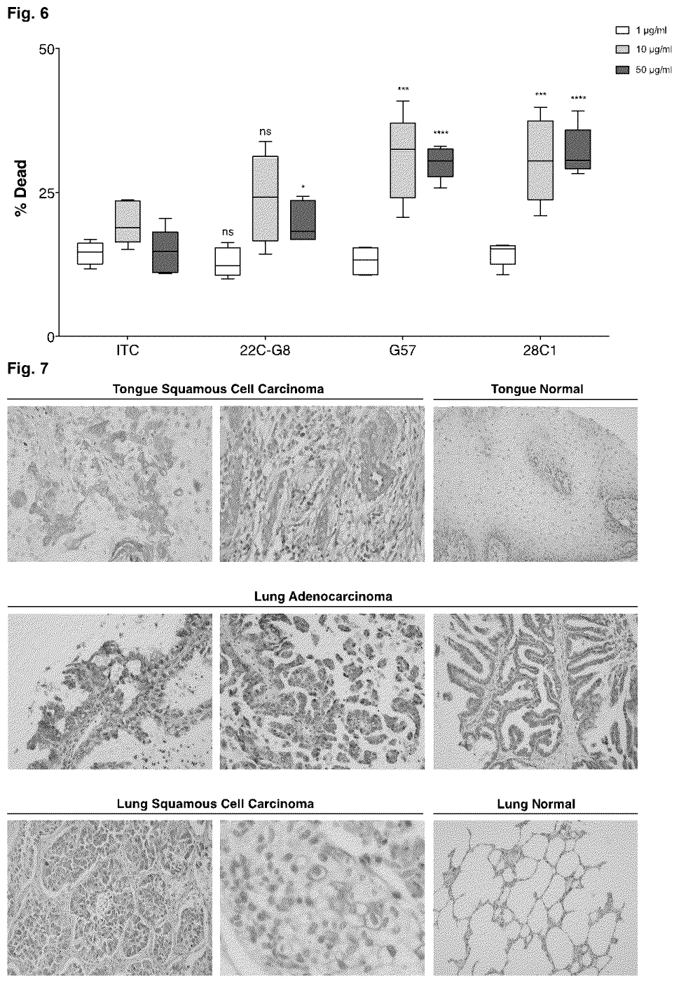

The inventors also addressed whether anti-LY6K antibodies were able to kill tumour cells through complement-dependent cytotoxicity. The inventors found that antibodies G57 and 28C1 were able to kill cervical cancer cells (Ca-Ski cells) at an antibody concentration of 10 and 50 μg/ml. Minimal killing was observed for 22C-G8, at a concentration of 50 μg/ml (FIG. 6).

### Example 6

In order for anti-LY6K antibodies to be used in cancer diagnosis (and treatment), antibody binding to healthy tissue should be minimal. Thus, the inventors performed immunohistochemistry on tongue squamous cell carcinoma (SCC), lung adenocarcinoma and lung SCC as well as healthy tongue and lung tissue sections (FIG. 7). The inventors found that anti-LY6K antibody candidate 28C1 stained malignant tongue and lung tissue but did not stain respective healthy tissues, showing that 28C1 is able to differentiate between malignant and healthy tissue samples.

### Example 7

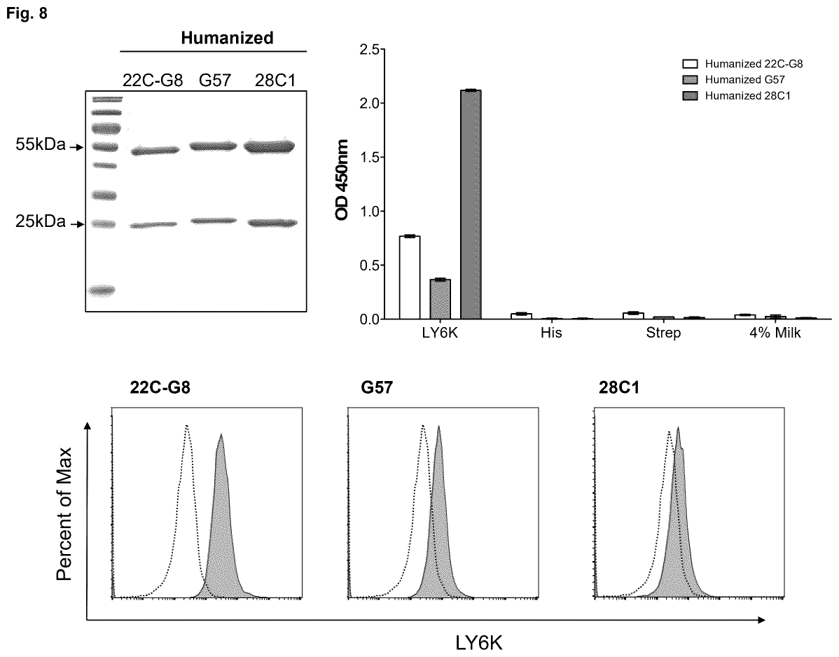

The murine antibodies were humanized through grafting of complementarity-determining regions (CDRs) into human framework regions. Humanized antibodies were expressed, and their purity was checked by SDS-PAGE (FIG. 8). The binding capacity of the humanized antibodies to recombinant LY6K was performed by ELISA assay. While humanized 22C-G8 and 28C1 revealed similar binding capacity towards recombinant LY6K as murine versions, humanized G57 showed reduced binding affinity to LY6K. Flow cytometry revealed that humanized 22C-G8 retained its strong binding capacity to Calu-1 cells, while humanized versions of G57 and 28C1 lost some of their affinity.

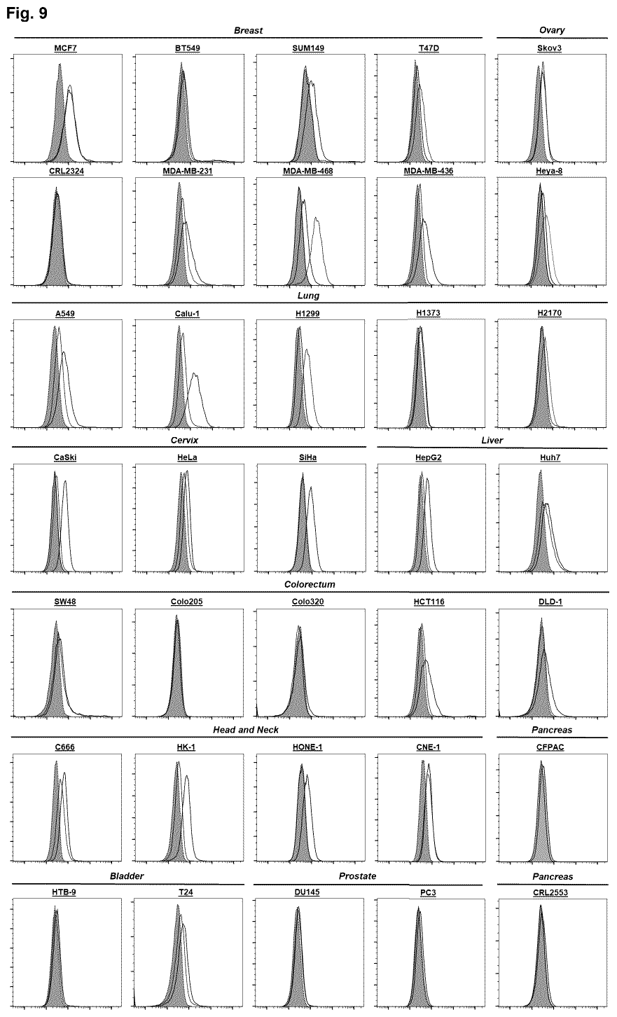

The inventors also validated the binding capacity of the humanized antibodies LY6K expressed on the surface of cancer cells, and confirmed that both humanized antibodies retained binding to LY6K expressed on the surface of a panel of human tumour cell lines derived from various origins including breast, cervix, lung and head and neck (FIG. 9).

### Example 8

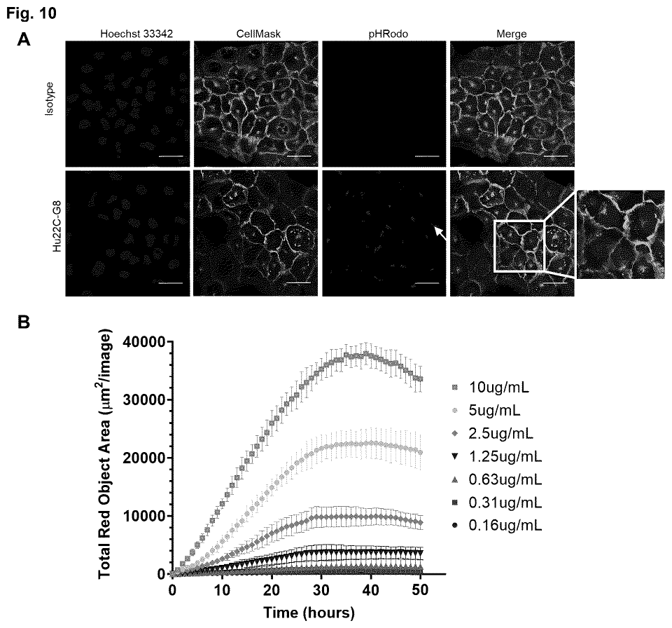

To further explore the potential of a humanized anti-LY6K antibody as an antibody-drug conjugate, the inventors tested if the antibody can be internalized. The inventors found that 22C-G8 antibody is internalized into Ca Ski cells, as shown by the punctate distribution of fluorescence signal in microscopy images, indicative of endosomal localization (FIG. 10A). In addition, real-time monitoring of the fluorescence intensity over time shows that the internalization is concentration- and time-dependent (FIG. 10B). As antibody internalization is a prerequisite for the development of an antibody into an antibody-drug conjugate, anti-LY6K antibodies show great potential as an antibody-drug conjugate for the delivery of cytotoxic drugs to LY6K-positive malignant cells.

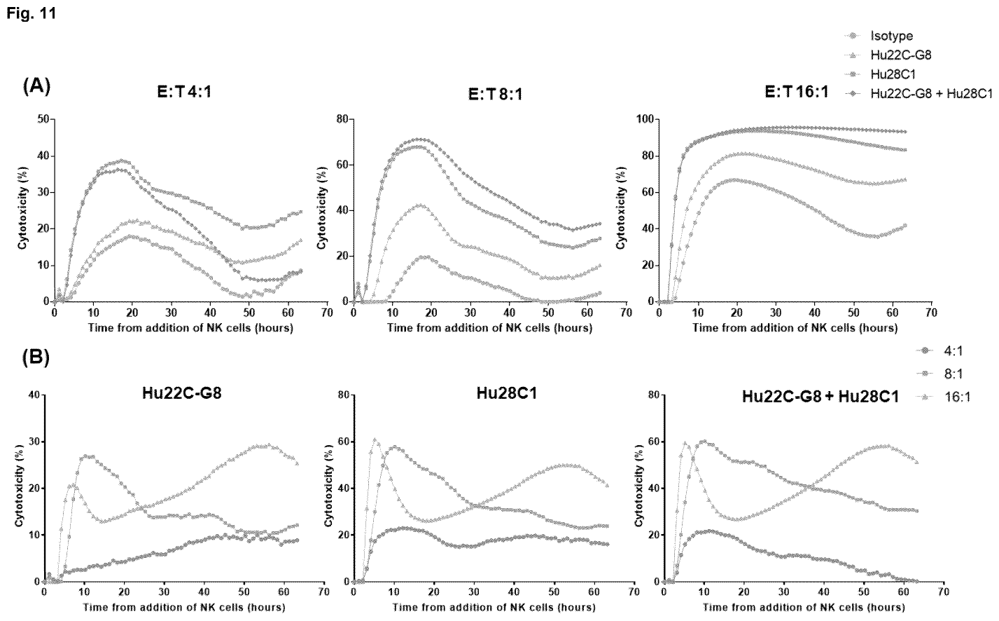

As antibody dependent cellular cytotoxicity (ADCC) is a crucial mechanism underlying targeted antibody-based immunotherapy approaches, the inventors investigated the potential of Hu22C-G8 and Hu28C1 in targeting and killing of LY6K expressing cells through ADCC (FIG. 11). The results show that in LY6K high expressing cells, both humanized antibodies can mediate ADCC and effectively eliminate tumour cells, with Hu28C1 showing higher cytotoxicity. This finding is significant as it suggests that LY6K expressed on tumour cells can be targeted by monoclonal antibodies in the presence of immune effector cells.

TABLE 1
Amino acid and nucleotide sequences of murine and humanized light and
heavy chain variable fragments of anti-LY6K antibody candidates.
CDR sequences underlined.

| | | SEQ ID |
|---|---|---|
| Antibody | Amino acid and Nucleotide sequence | NO: |
| Ms 22C-G8 L | KSSQSLLASDGKTYLN | 1 |
| CDR1 | | |
| Ms 22C-G8 L | LVSKLDS | 2 |
| CDR2 | | |
| Ms 22C-G8 L | WQGSHFPLT | 3 |
| CDR3 | | |
| Ms 22C-G8 L | DVVMTQSPLTLSVTIGQPASVSCKSSQSLLASDGKTYLN | 7 |
| | WLLQRPGQSPKRLIYLVSKLDSGVPDRFTGSGSGTDFTL | |
| | SISRVEAEDLGVYYCWQGSHFPLTFGPGTQLE | |
| Ms 22C-G8 L | GATGTTGTGATGACCCAGTCTCCACTCACTTTGTCGGT | 9 |
| | TACCATTGGACAACCAGCCTCCGTCTCTTGCAAGTCCA | |
| | GTCAGAGCCTCTTAGCCAGTGATGGAAAGACATATTTG | |
| | AATTGGTTGTTACAGAGGCCAGGCCAGTCTCCAAAGC | |
| | GCCTAATCTATCTGGTGTCTAAACTGGACTCTGGAGTC | |
| | CCTGACAGGTTCACTGGCAGTGGATCCGGGACAGATT | |
| | TCACACTGTCAATCAGCAGAGTGGAGGCTGAGGATTT | |
| | GGGAGTTTATTATTGCTGGCAAGGTTCACATTTTCCGC | |
| | TCACGTTCGGTCCTGGGACCCAGCTGGAG | |
| Ms 22C-G8 H | SYWMH | 4 |
| CDR1 | | |
| Ms 22C-G8 H | EINPSNGGANYNEKFKR | 5 |
| CDR2 | | |
| Ms 22C-G8 H | GGNFFFAY | 6 |
| CDR3 | | |
| Ms 22C-G8 H | QVQLQQPGAELAKPGASVKLSCKTSGYTFTSYWMHWV | 8 |
| | KQRPGQGFEWIGEINPSNGGANYNEKFKRKATLTVDKS | |
| | SSTAYMQLRSLTSEDSAVYYCTMGGNFFFAYWGQGTSV | |
| | TVSS | |
| Ms 22C-G8 H | CAGGTCCAACTGCAGCAGCCTGGGGCTGAACTGGCG | 10 |
| | AAGCCTGGGGCTTCAGTGAAGTTGTCCTGCAAGACCT | |
| | CTGGCTACACCTTCACCAGCTACTGGATGCACTGGGT | |
| | GAAGCAGAGGCCTGGACAAGGCTTTGAGTGGATTGGA | |
| | GAGATTAATCCTAGCAATGGTGGTGCTAACTACAATGA | |
| | GAAGTTCAAGAGAAAGGCCACACTGACTGTAGACAAA | |
| | TCTTCCAGCACAGCCTACATGCAACTCAGAAGCCTGA | |
| | CATCTGAGGACTCTGCGGTCTATTACTGTACAATGGG | |
| | GGGTAACTTCTTCTTTGCTTATTGGGGCCAAGGAACCT | |
| | CAGTCACCGTCTCGAGC | |
| Ms G57 L | RSSQSLVHTDGDTYLN | 11 |
| CDR1 | | |
| Ms G57 L | KVSNRFS | 12 |
| CDR2 | | |
| Ms G57 L | SQNTHVYT | 13 |
| CDR3 | | |
| Ms G57 L | DVVMTQTPLSLPVSLGDQASISCRSSQSLVHTDGDT | 17 |
| | YLNWYLQKPGQSPKLLIYKVSNRFSGVPDRFSGSGS | |
| | GTDFTLKIRRVEAEDLGVYFCSQNTHVYTFGGGTKL | |
| | E | |
| Ms G57 L | GATGTTGTGATGACCCAAACTCCACTCTCCCTGCCTGT | 19 |
| | CAGTCTTGGAGATCAAGCCTCCATCTCTTGCAGATCTA | |
| | GTCAGAGTCTTGTACATACTGATGGAGACACCTATTTA | |
| | AATTGGTACCTGCAGAAGCCGGGCCAGTCTCCAAAGC | |
| | TCCTGATCTACAAAGTTTCCAACCGATTTTCTGGGGTC | |
| | CCAGACAGGTTCAGTGGCAGTGGATCAGGGACAGATT | |
| | TCACACTCAAGATCAGGAGAGTGGAGGCTGAGGATCT | |
| | GGGAGTTTATTTCTGCTCTCAAAATACACATGTTTACA | |
| | CGTTCGGAGGGGGGACCAAGCTGGAA | |
| Ms G57 H | SYGMS | 14 |
| CDR1 | | |
| Ms G57 H | TISSGGTYTYYSDSVKG | 15 |
| CDR2 | | |
| Ms G57 H | HDWDFFDY | 16 |
| CDR3 | | |
| Ms G57 H | EVQLVESGGDLVKPGGSLKLSCAASGFTFNSYGMS | 18 |
| | WRQTPDKRLEWVATISSGGTYTYYSDSVKGRFTIS | |
| | RDNAKNILYLHMSSLKSEDTAMYYCARHDWDFFDY | |
| | WGQGTTLTVSS | |
| Ms G57 H | GAGGTGCAGCTTGTTGAGTCTGGGGGAGACTTAGTGA | 20 |
| | AGCCTGGAGGGTCCCTAAAACTCTCCTGTGCAGCCTC | |
| | TGGATTCACTTTCAATAGCTATGGCATGTCTTGGGTTC | |
| | GCCAGACTCCAGACAAGAGGCTAGAGTGGGTCGCAA | |
| | CCATTAGTAGTGGTGGTACTTACACATACTATTCAGAC | |
| | AGTGTGAAGGGGCGATTCACCATCTCCAGAGACAATG | |
| | CCAAGAATATTTTGTACCTGCACATGAGCAGTCTGAAG | |
| | TCTGAGGACACAGCCATGTATTACTGTGCAAGACATGA | |
| | TTGGGACTTCTTTGACTACTGGGGCCAAGGCACCACT | |
| | CTCACCGTCTCGAGC | |
| Ms 28C1 L | CRSSQTILHSNGNTYLE | 21 |
| CDR1 | | |
| Ms 28C1 L | KVSNRLS | 22 |
| CDR2 | | |
| Ms 28C1 L | FQGSHDPFT | 23 |
| CDR3 | | |
| Ms 28C1 L | DIQMTQTPLSLPVSLGDQASISCRSSQTILHSNGNTYLEW | 27 |
| | YLQKPGQSLKLLIYKVSNRLSGVPDRFSGSGSGTDFTLKI | |
| | SRVEAEDLGVYYCFQGSHDPFTFGSGTKLE | |
| Ms 28C1 L | GATATCCAGATGACACAGACTCCACTCTCCCTGCCTGT | 29 |
| | CAGTCTTGGAGATCAAGCCTCCATCTCTTGCAGATCTA | |
| | GTCAGACCATTTTACATAGTAATGGAAACACCTATTTA | |
| | GAATGGTACCTGCAGAAACCAGGCCAGTCTCTAAAAC | |
| | TCCTGATCTACAAAGTTTCCAACCGATTGTCTGGGGTC | |
| | CCAGACAGGTTCAGTGGCAGTGGATCAGGGACAGATT | |
| | TCACACTTAAGATCAGCAGAGTGGAGGCTGAGGATTT | |
| | GGGAGTTTATTACTGTTTTCAAGGTTCACATGATCCAT | |
| | TCACGTTCGGCTCGGGGACAAAGTTGGAA | |
| Ms 28C1 H | NFAMN | 24 |
| CDR1 | | |
| Ms 28C1 H | WINTNTGEPTYVEEFKG | 25 |
| CDR2 | | |
| Ms 28C1 H | REWRRSYYFDY | 26 |
| CDR3 | | |
| Ms 28C1 H | QIQLVQSGPELKKPGETVKISCKTSGYTFTNFAMNW | 28 |
| | VKQTPGKGLRYMGWINTNTGEPTYVEEFKGRFAFSL | |
| | ETSASTAYLQISNLKNEDTATYFCTREWRRSYYFDY | |
| | WGQGTSLTVSS | |
| Ms 28C1 H | CAGATCCAGTTGGTGCAGTCTGGACCTGAGCTGAAGA | 30 |
| | AGCCTGGCGAGACAGTCAAGATCTCCTGCAAGACTTC | |
| | TGGATATACCTTCACAAACTTTGCAATGAACTGGGTGA | |
| | AGCAGACTCCAGGAAAGGGTTTAAGGTACATGGGCTG | |
| | GATAAACACCAACACTGGAGAGCCAACATATGTTGAA | |
| | GAGTTCAAGGGACGGTTTGCCTTCTCTTTGGAAACCTC | |
| | TGCCAGCACTGCCTATTTGCAGATCAGCAACCTCAAAA | |
| | ATGAGGACACGGCTACATATTTCTGTACAAGAGAGTG | |
| | GAGGAGGTCATACTACTTTGACTACTGGGGCCAAGGC | |
| | ACCTCTCTCACCGTCTCGAGC | |
| Hu 22C-G8 L | DVVMTQSPLSLSVTLGQPASISCKSSQSLLASDGKT | 31 |
| | YLNWFQQRPGQSPRRLIYLVSKLDSGVPDRFSGSG | |
| | SGTDFTLKISRVEAEDVGVYYCWQGSHFPLTFGGGT | |
| | KVE | |
| Hu 22C-G8 L | GATGTTGTGATGACTCAGTCTCCACTCTCCCTGTCGGT | 33 |
| | CACCCTTGGACAGCCGGCCTCCATCTCCTGCAAGTCC | |
| | AGTCAGAGCCTCTTAGCCAGTGATGGAAAGACATATTT | |
| | GAATTGGTTTCAGCAGAGGCCAGGCCAATCTCCAAGG | |
| | CGCCTAATTTATCTGGTGTCTAAACTGGACTCTGGGGT | |
| | CCCAGACAGATTCAGCGGCAGTGGGTCAGGCACTGAT | |
| | TTCACACTGAAAATCAGCAGGGTGGAGGCTGAGGATG | |
| | TTGGGGTTTATTACTGCTGGCAAGGTTCACATTTTCCG | |
| | CTCACGTTCGGCGGAGGGACCAAGGTGGAG | |
| Hu 22C-G8 H | QVQLVQSGAEVKKPGASVKVSCKASGYTFTSYWMHWV | 32 |
| | RQAPGQGLEWIGEINPSNGGANYNEKFKRRATMTVDKS | |
| | TSTVYMELSSLRSEDTAVYYCTMGGNFFFAYWGQGTLV | |
| | TVSS | |
| Hu 22C-G8 H | CAGGTGCAGCTGGTGCAGTCTGGGGCTGAGGTGAAG | 34 |
| | AAGCCTGGGGCCTCAGTGAAGGTTTCCTGCAAGGCAT | |
| | CTGGCTACACCTTCACCAGCTACTGGATGCACTGGGT | |
| | GCGACAGGCCCCTGGACAAGGGCTTGAGTGGATTGG | |
| | AGAGATTAATCCTAGCAATGGTGGTGCTAACTACAATG | |
| | AGAAGTTCAAGAGAAGAGCCACCATGACCGTAGACAA | |
| | ATCCACGAGCACAGTCTACATGGAGCTGAGCAGCCTG | |
| | AGATCTGAGGACACGGCCGTGTATTACTGTACAATGG | |
| | GGGGTAACTTCTTCTTTGCTTATTGGGGCCAAGGAAC | |
| | CCTGGTCACCGTCTCGAGC | |
| Hu G57 L | DVVMTQSPLSLPVTLGQPASISCRSSQSLVHTDGDTYLN | 35 |
| | WFQQRPGQSPRRLIYKVSNRFSGVPDRFSGSGSGTDFT | |
| | LKISRVEAEDVGVYYCSQNTHVYTFGQGTKLE | |
| Hu G57 L | GATGTTGTGATGACTCAGTCTCCACTCTCCCTGCCCGT | 40 |
| | CACCCTTGGACAGCCGGCCTCCATCTCCTGCAGATCT | |
| | AGTCAGAGTCTTGTACATACTGATGGAGACACCTATTT | |
| | AAATTGGTTTCAGCAGAGGCCAGGCCAATCTCCAAGG | |
| | CGCCTAATTTATAAAGTTTCCAACCGATTTTCTGGGGT | |
| | CCCAGACAGATTCAGCGGCAGTGGGTCAGGCACTGAT | |
| | TTCACACTGAAAATCAGCAGGGTGGAGGCTGAGGATG | |
| | TTGGGGTTTATTACTGCTCTCAAAATACACATGTTTA | |
| | CACGTTTGGCCAGGGGACCAAGCTGGAG | |
| Hu G57 H | EVQLVESGGGLVKPGGSLRLSCAASGFTFNSYGMS | 38 |
| | WRQAPGKRLEWVSTISSGGTYTYYSDSVKGRFTIS | |
| | RDNAKNSLYLQMNSLRAEDTAVYYCARHDWDFFDY | |
| | WGQGTLVTVSS | |
| Hu G57 H | GAGGTGCAGCTGGTGGAGTCTGGGGGAGGCCTGGTC | 43 |
| | AAGCCTGGGGGGTCCCTGAGACTCTCCTGTGCAGCCT | |
| | CTGGATTCACCTTCAATAGCTATGGCATGTCTTGGGT | |
| | CCGCCAGGCTCCAGGGAAGAGGCTGGAGTGGGTCTCA | |
| | ACCATTAGTAGTGGTGGTACTTACACATACTATTCAGA | |
| | CAGTGTGAAGGGGCGATTCACCATCTCCAGAGACAAC | |
| | GCCAAGAACTCACTGTATCTGCAAATGAACAGCCTGA | |
| | GAGCCGAGGACACGGCTGTGTATTACTGTGCGAGACA | |
| | TGATTGGGACTTCTTTGACTACTGGGGCCAAGGAACC | |
| | CTGGTCACCGTCTCGAGC | |
| Hu G57 L V2 | DVVMTQSPLSLPVTLGQPASISCRSSQSLVHTDGDT | 36 |
| | YLNWYQQRPGQSPRRLIYKVSNRFSGVPDRFSGSG | |
| | SGTDFTLKISRVEAEDVGVYYCSQNTHVYTFGQGTK | |
| | LE | |
| Hu G57 L V2 | GATGTTGTGATGACTCAGTCTCCACTCTCCCTGCCCGT | 41 |
| | CACCCTTGGACAGCCGGCCTCCATCTCCTGCAGATCT | |
| | AGTCAGAGTCTTGTACATACTGATGGAGACACCTATTT | |
| | AAATTGGTACCAGCAGAGGCCAGGCCAATCTCCAAGG | |
| | CGCCTAATTTATAAAGTTTCCAACCGATTTTCTGGGGT | |
| | CCCAGACAGATTCAGCGGCAGTGGGTCAGGCACTGAT | |
| | TTCACACTGAAAATCAGCAGGGTGGAGGCTGAGGATG | |
| | TTGGGGTTTATTACTGCTCTCAAAATACACATGTTTA | |
| | CACGTTTGGCCAGGGGACCAAGCTGGAG | |
| Hu G57 H V2 | EVQLVESGGGLVKPGGSLRLSCAASGFTFNSYGMS | 39 |
| | WRQAPGKRLEWATISSGGTYTYYSDSVKGRFTIS | |
| | RDNAKNSLYLQMNSLRAEDTAVYYCARHDWDFFDY | |
| | WGQGTLVTVSS | |
| Hu G57 H V2 | GAGGTGCAGCTGGTGGAGTCTGGGGGAGGCCTGGTC | 44 |
| | AAGCCTGGGGGGTCCCTGAGACTCTCCTGTGCAGCCT | |
| | CTGGATTCACCTTCAATAGCTATGGCATGTCTTGGGT | |
| | CCGCCAGGCTCCAGGGAAGAGGCTGGAGTGGGTCGCA | |
| | ACCATTAGTAGTGGTGGTACTTACACATACTATTCAGA | |
| | CAGTGTGAAGGGGCGATTCACCATCTCCAGAGACAAC | |
| | GCCAAGAACTCACTGTATCTGCAAATGAACAGCCTGA | |
| | GAGCCGAGGACACGGCTGTGTATTACTGTGCGAGACA | |
| | TGATTGGGACTTCTTTGACTACTGGGGCCAAGGAACC | |
| | CTGGTCACCGTCTCGAGC | |
| Hu G57 L V3 | DWVMTQSPLSLPVTLGQPASISCRSSQSLVHTDGDTYLN | 37 |
| | WYQQRPGQSPRRLIYKVSNRFSGVPDRFSGSGSGTDFT | |
| | LKISRVEAEDVGVYFCSQNTHVYTFGQGTKLE | |
| Hu G57 L V3 | GATGTTGTGATGACTCAGTCTCCACTCTCCCTGCCCGT | 42 |
| | CACCCTTGGACAGCCGGCCTCCATCTCCTGCAGATCT | |
| | AGTCAGAGTCTTGTACATACTGATGGAGACACCTATTT | |
| | AAATTGGTACCAGCAGAGGCCAGGCCAATCTCCAAGG | |
| | CGCCTAATTTATAAAGTTTCCAACCGATTTTCTGGGGT | |
| | CCCAGACAGATTCAGCGGCAGTGGGTCAGGCACTGAT | |
| | TTCACACTGAAAATCAGCAGGGTGGAGGCTGAGGATGT | |
| | TGGGGTTTATTTCTGCTCTCAAAATACACATGTTTACA | |
| | CGTTTGGCCAGGGGACCAAGCTGGAG | |
| Hu 28C1 L | DVVMTQSPLSLPVTLGQPASISCRSSQTILHSNGNTYLEW | 45 |
| | YQQRPGQSPRLLIYKVSNRLSGVPDRFSGSGSGTDFTLKI | |
| | SRVEAEDVGVYYCFQGSHDPFTFGPGTKVD | |
| Hu 28C1 L | GATGTTGTGATGACTCAGTCTCCACTCTCCCTGCCCGT | 49 |
| | CACCCTTGGACAGCCGGCCTCCATCTCCTGCAGATCTA | |
| | GTCAGACCATTTTACATAGTAATGGAAACACCTATTTA | |
| | GAATGGTACCAGCAGAGGCCAGGCCAATCTCCAAGGC | |
| | TCCTAATTTATAAAGTTTCCAACCGATTGTCTGGGGTC | |
| | CCAGACAGATTCAGCGGCAGTGGGTCAGGCACTGATT | |
| | TCACACTGAAAATCAGCAGGGTGGAGGCTGAGGATGT | |
| | TGGGGTTTATTACTGCTTTCAAGGTTCACATGATCCAT | |
| | TCACGTTCGGCCCTGGGACCAAAGTGGAT | |
| Hu 28C1 H | QVQLVQSGSELKKPGASVKVSCKASGYTFTNFAMNWVR | 47 |
| | QAPGQGLEYMGWINTNTGEPTYVEEFKGRFVFSLDTSV | |
| | STAYLQISSLKAEDTAVYYCTREWRRSYYFDYWGQGTL | |
| | VTVSS | |
| Hu 28C1 H | CAGGTGCAGCTGGTGCAATCTGGGTCTGAGTTGAAGA | 51 |
| | AGCCTGGGGCCTCAGTGAAGGTTTCCTGCAAGGCTTC | |
| | TGGATACACCTTCACTAACTTTGCAATGAACTGGGTG | |
| | CGACAGGCCCCTGGACAAGGGCTTGAGTACATGGGAT | |
| | GGATAAACACCAACACTGGAGAGCCAACATATGTTGA | |
| | AGAGTTCAAGGGACGGTTTGTCTTCTCCTTGGACACC | |
| | TCTGTCAGCACGGCATATCTGCAGATCAGCAGCCTAA | |
| | AGGCTGAGGACACTGCCGTGTATTACTGTACAAGAGA | |
| | GTGGAGGAGGTCATACTACTTTGACTACTGGGGCCAA | |
| | GGAACCCTGGTCACCGTCTCGAGC | |
| Hu 28C1 L V2 | DVVMTQSPLSLPVTLGQPASISCRSSQTILHSNGNTY | 46 |
| | LEWYQQRPGQSLRLLIYKVSNRLSGVPDRFSGSGSGT | |
| | DFTLKISRVEAEDVGVYYCFQGSHDPFTFGPGTKVD | |
| Hu 28C1 L V2 | CAGGTGCAGCTGGTGCAATCTGGGTCTGAGTTGAAGA | 50 |
| | AGCCTGGGGCCTCAGTGAAGGTTTCCTGCAAGGCTTC | |
| | TGGATACACCTTCACTAACTTTGCAATGAACTGGGTG | |
| | CGACAGGCCCCTGGACAAGGGCTTAGGTACATGGGAT | |
| | GGATAAACACCAACACTGGAGAGCCAACATATGTTGA | |
| | AGAGTTCAAGGGACGGTTTGTCTTCTCCTTGGACACCT | |
| | CTGTCAGCACGGCATATCTGCAGATCAGCAGCCTAAA | |
| | GGCTGAGGACACTGCCGTGTATTACTGTACAAGAGAG | |
| | TGGAGGAGGTCATACTACTTTGACTACTGGGGCCAAG | |
| | GAACCCTGGTCACCGTCTCGAGC | |
| Hu 28C1 H V2 | QVQLVQSGSELKKPGASVKVSCKASGYTFTNFAMNWRQ | 48 |
| | APGQGLRYMGWINTNTGEPTYVEEFKGRFVFSLDTSVS | |
| | TAYLQISSLKAEDTAVYYCTREWRRSYYFDYWGQGTLV | |
| | TVSS | |
| Hu 28C1 H V2 | GATGTTGTGATGACTCAGTCTCCACTCTCCCTGCCCGT | 52 |
| | CACCCTTGGACAGCCGGCCTCCATCTCCTGCAGATCTA | |
| | GTCAGACCATTTTACATAGTAATGGAAACACCTATTTA | |
| | GAATGGTACCAGCAGAGGCCAGGCCAATCTCTAAGGC | |
| | TCCTAATTTATAAAGTTTCCAACCGATTGTCTGGGGTC | |
| | CCAGACAGATTCAGCGGCAGTGGGTCAGGCACTGATT | |
| | TCACACTGAAAATCAGCAGGGTGGAGGCTGAGGATGT | |
| | TGGGGTTTATTACTGCTTTCAAGGTTCACATGATCCAT | |
| | TCACGTTCGGCCCTGGGACCAAAGTGGAT | |

### SUMMARY

The inventors found that LYSK is a CTA that is expressed on the cell surface of various human cancers and therefore represents an attractive target antigen for antibody-based approaches. The inventors developed three unique anti-LYSK antibodies and provide evidence that they have the potential to be useful for cancer diagnosis and/or treatment.

### REFERENCES

Any listing or discussion of an apparently prior-published document in this specification should not necessarily be taken as an acknowledgement that such document is part of the state of the art or is common general knowledge.
1. Chalouni, C., Doll, S. Fate of Antibody-Drug Conjugates in Cancer Cells. J Exp Clin Cancer Res 37, 20 (2018).
2. Langer R. New methods of drug delivery. Science. 249 (4976): 1527-33 (1990).
3. Remington J. P. and Gennaro A. R. The Science and Practice of Pharmacy, 19^{th} ed., Easton, PA: Mack Publishing (1995).
4. Sambrook and Russel, Molecular Cloning: A Laboratory Manual, Cold Springs Harbor Laboratory, New York (2001).

1. An isolated antibody or a fragment thereof that specifically binds to at least one epitope of Lymphocyte antigen 6 family member K (LY6K), wherein the antibody or fragment thereof comprises at least one variable light chain and at least one variable heavy chain, wherein:
a) the variable light chain amino acid sequence comprises six complementarity determining regions (CDRs), wherein the light chain CDR1 comprises the amino acid sequence KSSQSLLASDGKTYLN set forth in SEQ ID NO: 1; the light chain CDR2 comprises the amino acid sequence LVSKLDS set forth in SEQ ID NO: 2; the light chain CDR3 comprises the amino acid sequence WQGSHFPLT set forth in SEQ ID NO: 3; the heavy chain CDR1 comprises the amino acid sequence SYWMH set forth in SEQ ID NO: 4; the heavy chain CDR2 comprises the amino acid sequence EINPSNGGANYNEKFKR set forth in SEQ ID NO: 5; and the heavy chain CDR3 comprises the amino acid sequence GGNFFFAY set forth in SEQ ID NO: 6; or
b) the variable light chain amino acid sequence comprises six complementarity determining regions (CDRs), wherein the light chain CDR1 comprises the amino acid sequence RSSQSLVHTDGDTYLN set forth in SEQ ID NO: 11; the light chain CDR2 comprises the amino acid sequence KVSNRFS set forth in SEQ ID NO: 12; the light chain CDR3 comprises the amino acid sequence SQNTHVYT set forth in SEQ ID NO: 13; the heavy chain CDR1 comprises the amino acid sequence SYGMS set forth in SEQ ID NO: 14; the heavy chain CDR2 comprises the amino acid sequence TISSGGTYTYYSDSVKG set forth in SEQ ID NO: 15; and the heavy chain CDR3 comprises the amino acid sequence HDWDFFDY set forth in SEQ ID NO: 16; or
c) the variable light chain amino acid sequence comprises six complementarity determining regions (CDRs), wherein the light chain CDR1 comprises the amino acid sequence CRSSQTILHSNGNTYLE set forth in SEQ ID NO: 21; the light chain CDR2 comprises the amino acid sequence KVSNRLS set forth in SEQ ID NO: 22; the light chain CDR3 comprises the amino acid sequence FQGSHDPFT set forth in SEQ ID NO: 23; the heavy chain CDR1 comprises the amino acid sequence NFAMN set forth in SEQ ID NO: 24; the heavy chain CDR2 comprises the amino acid sequence WINTNTGEPTYVEEFKG set forth in SEQ ID NO: 25; and the heavy chain CDR3 comprises the amino acid sequence REWRRSYYFDY set forth in SEQ ID NO: 26.

2. The isolated antibody or a fragment thereof according to claim 1, wherein:
a) the light chain comprises an amino acid sequence selected from the group comprising SEQ ID NO: 7 and SEQ ID NO: 31, and the variable heavy chain comprises an amino acid sequence selected from the group comprising SEQ ID NO: 8 and SEQ ID NO: 32;
b) the light chain comprises an amino acid sequence selected from the group comprising SEQ ID NO: 17, SEQ ID NO: 35, SEQ ID NO: 36 and SEQ ID NO: 37, and the variable heavy chain comprises an amino acid sequence selected from the group comprising SEQ ID NO: 18, SEQ ID NO: 38 and SEQ ID NO: 39; or
c) the light chain comprises an amino acid sequence selected from the group comprising SEQ ID NO: 27, SEQ ID NO: 45 and SEQ ID NO: 46, and the variable heavy chain comprises an amino acid sequence selected from the group comprising SEQ ID NO: 28, SEQ ID NO: 47 and SEQ ID NO: 48.

3. The isolated antibody or fragment thereof according to claim 1, wherein said isolated antibody or fragment thereof is an antibody conjugate.

4. The isolated antibody or fragment thereof according to claim 3, wherein said isolated antibody or fragment thereof is conjugated to a cytotoxic drug, a radioactive moiety, or an identifiable moiety.

5. The isolated antibody or fragment thereof according to claim 1, wherein the antibody is a monoclonal mouse, humanized or chimeric antibody.

6. A pharmaceutical composition comprising the isolated antibody or fragment thereof defined in claim 1 and a pharmaceutical acceptable excipient, diluent, salt, or carrier.

7. An isolated antibody or fragment thereof according to claim 1, or a pharmaceutical composition according to claim 6, for the treatment of LY6K-positive malignant cells.

8. The isolated antibody, fragment thereof, or pharmaceutical composition according to claim 7, wherein the LY6K-positive malignant cells are selected from the group comprising cervical cancer, lung adenocarcinoma, lung squamous cell carcinoma (SCC), tongue SCC, head and neck, and breast cancer.

9. (canceled)

10. (canceled)

11. A method of treating a LY6K-positive malignant cell disease, the method comprising administering to a subject an antibody or fragment thereof defined in claim 1.

12. The method according to claim 11, wherein the LY6K-positive malignant cell disease is selected from the group consisting of cancer, such as cervical cancer, lung adenocarcinoma, lung squamous cell carcinoma (SCC), tongue SCC, head and neck, and breast cancer.

13. A method of detecting a LY6K-positive malignant cell in a biological sample, comprising:
i) providing at least one biological sample;
ii) contacting the at least one biological sample with an antibody or fragment thereof of claim 1;
iii) detecting specific binding; and
iv) comparing the binding in iii) with a control.

14. An isolated nucleic acid molecule encoding:
(a) at least one variable light chain of the antibody defined in claim 2, wherein the variable light chain comprises at least one amino acid sequence selected from the group consisting of SEQ ID NO: 7 and SEQ ID NO: 31, and
(b) at least one variable heavy chain of the antibody defined in claim 1, wherein the variable heavy chain comprises at least one amino acid sequence selected from the group consisting of SEQ ID NO: 8 and SEQ ID NO: 32; or
(c) at least one variable light chain of the antibody defined in claim 2, wherein the variable light chain comprises at least one amino acid sequence selected from the group consisting of SEQ ID NO: 17, SEQ ID NO: 35, SEQ ID NO: 36 and SEQ ID NO: 37, and
(d) at least one variable heavy chain of the antibody defined in claim 1, wherein the variable heavy chain comprises at least one amino acid sequence selected from the group consisting of SEQ ID NO: 18, SEQ ID NO: 38 and SEQ ID NO: 39; or
(e) at least one variable light chain of the antibody defined in claim 2, wherein the variable light chain comprises at least one amino acid sequence selected from the group consisting of SEQ ID NO: 27, SEQ ID NO: 45 and SEQ ID NO: 46, and
(f) at least one variable heavy chain of the antibody defined in claim 1, wherein the variable heavy chain comprises at least one amino acid sequence selected from the group consisting of SEQ ID NO: 28, SEQ ID NO: 47 and SEQ ID NO: 48.

15. The isolated nucleic acid molecule according to claim 14, wherein:
i) the at least one nucleic acid sequence in (a) has at least 80%, at least 90% or at least 95% sequence identity to SEQ ID NO: 9 and/or SEQ ID NO: 33; and the at least one nucleic acid sequence in (b) has at least 80%, at least 90% or at least 95% sequence identity to SEQ ID NO: 10 and/or SEQ ID NO: 34; or
ii) the nucleic acid sequence in (c) has at least 80%, at least 90% or at least 95% sequence identity to SEQ ID NO: 19, SEQ ID NO: 40, SEQ ID NO: 41 and/or SEQ ID NO: 42; and the at least one nucleic acid sequence in (d) has at least 80%, at least 90% or at least 95% sequence identity to SEQ ID NO: 20, SEQ ID NO: 43 and/or SEQ ID NO: 44; or
iii) the nucleic acid sequence in (e) has at least 80%, at least 90% or at least 95% sequence identity to SEQ ID NO: 29, SEQ ID NO: 49 and/or SEQ ID NO: 50; and the at least one nucleic acid sequence in (f) has at least 80%, at least 90% or at least 95% sequence identity to SEQ ID NO: 30, SEQ ID NO: 51 and/or SEQ ID NO: 52.

16. An expression vector comprising at least one isolated nucleic acid molecule defined in claim 14.

17. A host cell comprising the expression vector defined in claim 16.

18. A kit comprising at least one antibody or fragment thereof defined in claim 1.

19. The isolated antibody or fragment thereof according to claim 1, wherein said isolated fragment thereof is selected from the group comprising scFv, Fab, Fab′, F(ab′)2, and Fv fragments; diabodies; linear antibodies; single-chain antibody molecules; and multispecific antibodies formed from antibody fragments.

20. The isolated antibody or fragment thereof according to claim 2, wherein said isolated fragment thereof is selected from the group comprising scFv, Fab, Fab′, F(ab′)2, and Fv fragments; diabodies; linear antibodies; single-chain antibody molecules; and multispecific antibodies formed from antibody fragments.


SEQUENCE LISTING (52 sequences)

SEQ ID NO: 1 - protein, 16 aa, synthetic construct
KSSQSLLASDGKTYLN

SEQ ID NO: 2 - protein, 7 aa, synthetic construct
LVSKLDS

SEQ ID NO: 3 - protein, 9 aa, synthetic construct
WQGSHFPLT

SEQ ID NO: 4 - protein, 5 aa, synthetic construct
SYWMH

SEQ ID NO: 5 - protein, 17 aa, synthetic construct
EINPSNGGANYNEKFKR

SEQ ID NO: 6 - protein, 8 aa, synthetic construct
GGNFFFAY

SEQ ID NO: 7 - protein, 110 aa, synthetic construct
DVVMTQSPLTLSVTIGQPASVSCKSSQSLLASDGKTYLNWLLQRPGQSPKRLIYLVSKLDSGVPDRFTGSGSGTDFTLSISRVEAEDLGVYYCWQGSHFPLTFGPGTQLE

SEQ ID NO: 8 - protein, 117 aa, synthetic construct
QVQLQQPGAELAKPGASVKLSCKTSGYTFTSYWMHWVKQRPGQGFEWIGEINPSNGGANYNEKFKRKATLTVDKSSSTAYMQLRSLTSEDSAVYYCTMGGNFFFAYWGQGTSVTVSS

SEQ ID NO: 9 - DNA, 330 bp, synthetic construct
  source 1..330: mol_type = other DNA
gatgttgtgatgacccagtctccactcactttgtcggttaccattggacaaccagcctccgtctcttgcaagtccagtcagagcctcttagccagtgatggaaagacatatttgaattggttgttacagaggccaggccagtctccaaagcgcctaatctatctggtgtctaaactggactctggagtccctgacaggttcactggcagtggatccgggacagatttcacactgtcaatcagcagagtggaggctgaggatttgggagtttattattgctggcaaggttcacattttccgctcacgttcggtcctgggacccagctggag

SEQ ID NO: 10 - DNA, 351 bp, synthetic construct
  source 1..351: mol_type = other DNA
caggtccaactgcagcagcctggggctgaactggcgaagcctggggcttcagtgaagttgtcctgcaagacctctggctacaccttcaccagctactggatgcactgggtgaagcagaggcctggacaaggctttgagtggattggagagattaatcctagcaatggtggtgctaactacaatgagaagttcaagagaaaggccacactgactgtagacaaatcttccagcacagcctacatgcaactcagaagcctgacatctgaggactctgcggtctattactgtacaatggggggtaacttcttctttgcttattggggccaaggaacctcagtcaccgtctcgagc

SEQ ID NO: 11 - protein, 16 aa, synthetic construct
RSSQSLVHTDGDTYLN

SEQ ID NO: 12 - protein, 7 aa, synthetic construct
KVSNRFS

SEQ ID NO: 13 - protein, 8 aa, synthetic construct
SQNTHVYT

SEQ ID NO: 14 - protein, 5 aa, synthetic construct
SYGMS

SEQ ID NO: 15 - protein, 17 aa, synthetic construct
TISSGGTYTYYSDSVKG

SEQ ID NO: 16 - protein, 8 aa, synthetic construct
HDWDFFDY

SEQ ID NO: 17 - protein, 109 aa, synthetic construct
DVVMTQTPLSLPVSLGDQASISCRSSQSLVHTDGDTYLNWYLQKPGQSPKLLIYKVSNRFSGVPDRFSGSGSGTDFTLKIRRVEAEDLGVYFCSQNTHVYTFGGGTKLE

SEQ ID NO: 18 - protein, 117 aa, synthetic construct
EVQLVESGGDLVKPGGSLKLSCAASGFTFNSYGMSWVRQTPDKRLEWVATISSGGTYTYYSDSVKGRFTISRDNAKNILYLHMSSLKSEDTAMYYCARHDWDFFDYWGQGTTLTVSS

SEQ ID NO: 19 - DNA, 327 bp, synthetic construct
  source 1..327: mol_type = other DNA
gatgttgtgatgacccaaactccactctccctgcctgtcagtcttggagatcaagcctccatctcttgcagatctagtcagagtcttgtacatactgatggagacacctatttaaattggtacctgcagaagccgggccagtctccaaagctcctgatctacaaagtttccaaccgattttctggggtcccagacaggttcagtggcagtggatcagggacagatttcacactcaagatcaggagagtggaggctgaggatctgggagtttatttctgctctcaaaatacacatgtttacacgttcggaggggggaccaagctggaa

SEQ ID NO: 20 - DNA, 351 bp, synthetic construct
  source 1..351: mol_type = other DNA
gaggtgcagcttgttgagtctgggggagacttagtgaagcctggagggtccctaaaactctcctgtgcagcctctggattcactttcaatagctatggcatgtcttgggttcgccagactccagacaagaggctagagtgggtcgcaaccattagtagtggtggtacttacacatactattcagacagtgtgaaggggcgattcaccatctccagagacaatgccaagaatattttgtacctgcacatgagcagtctgaagtctgaggacacagccatgtattactgtgcaagacatgattgggacttctttgactactggggccaaggcaccactctcaccgtctcgagc

SEQ ID NO: 21 - protein, 17 aa, synthetic construct
CRSSQTILHSNGNTYLE

SEQ ID NO: 22 - protein, 7 aa, synthetic construct
KVSNRLS

SEQ ID NO: 23 - protein, 9 aa, synthetic construct
FQGSHDPFT

SEQ ID NO: 24 - protein, 5 aa, synthetic construct
NFAMN

SEQ ID NO: 25 - protein, 17 aa, synthetic construct
WINTNTGEPTYVEEFKG

SEQ ID NO: 26 - protein, 11 aa, synthetic construct
REWRRSYYFDY

SEQ ID NO: 27 - protein, 110 aa, synthetic construct
DIQMTQTPLSLPVSLGDQASISCRSSQTILHSNGNTYLEWYLQKPGQSLKLLIYKVSNRLSGVPDRFSGSGSGTDFTLKISRVEAEDLGVYYCFQGSHDPFTFGSGTKLE

SEQ ID NO: 28 - protein, 119 aa, synthetic construct
QIQLVQSGPELKKPGETVKISCKTSGYTFTNFAMNWVKQTPGKGLRYMGWINTNTGEPTYVEEFKGRFAFSLETSASTAYLQISNLKNEDTATYFCTREWRRSYYFDYWGQGTSLTVSS

SEQ ID NO: 29 - DNA, 330 bp, synthetic construct
  source 1..330: mol_type = other DNA
gatatccagatgacacagactccactctccctgcctgtcagtcttggagatcaagcctccatctcttgcagatctagtcagaccattttacatagtaatggaaacacctatttagaatggtacctgcagaaaccaggccagtctctaaaactcctgatctacaaagtttccaaccgattgtctggggtcccagacaggttcagtggcagtggatcagggacagatttcacacttaagatcagcagagtggaggctgaggatttgggagtttattactgttttcaaggttcacatgatccattcacgttcggctcggggacaaagttggaa

SEQ ID NO: 30 - DNA, 357 bp, synthetic construct
  source 1..357: mol_type = other DNA
cagatccagttggtgcagtctggacctgagctgaagaagcctggcgagacagtcaagatctcctgcaagacttctggatataccttcacaaactttgcaatgaactgggtgaagcagactccaggaaagggtttaaggtacatgggctggataaacaccaacactggagagccaacatatgttgaagagttcaagggacggtttgccttctctttggaaacctctgccagcactgcctatttgcagatcagcaacctcaaaaatgaggacacggctacatatttctgtacaagagagtggaggaggtcatactactttgactactggggccaaggcacctctctcaccgtctcgagc

SEQ ID NO: 31 - protein, 110 aa, synthetic construct
DVVMTQSPLSLSVTLGQPASISCKSSQSLLASDGKTYLNWFQQRPGQSPRRLIYLVSKLDSGVPDRFSGSGSGTDFTLKISRVEAEDVGVYYCWQGSHFPLTFGGGTKVE

SEQ ID NO: 32 - protein, 117 aa, synthetic construct
QVQLVQSGAEVKKPGASVKVSCKASGYTFTSYWMHWVRQAPGQGLEWIGEINPSNGGANYNEKFKRRATMTVDKSTSTVYMELSSLRSEDTAVYYCTMGGNFFFAYWGQGTLVTVSS

SEQ ID NO: 33 - DNA, 330 bp, synthetic construct
  source 1..330: mol_type = other DNA
gatgttgtgatgactcagtctccactctccctgtcggtcacccttggacagccggcctccatctcctgcaagtccagtcagagcctcttagccagtgatggaaagacatatttgaattggtttcagcagaggccaggccaatctccaaggcgcctaatttatctggtgtctaaactggactctggggtcccagacagattcagcggcagtgggtcaggcactgatttcacactgaaaatcagcagggtggaggctgaggatgttggggtttattactgctggcaaggttcacattttccgctcacgttcggcggagggaccaaggtggag

SEQ ID NO: 34 - DNA, 351 bp, synthetic construct
  source 1..351: mol_type = other DNA
caggtgcagctggtgcagtctggggctgaggtgaagaagcctggggcctcagtgaaggtttcctgcaaggcatctggctacaccttcaccagctactggatgcactgggtgcgacaggcccctggacaagggcttgagtggattggagagattaatcctagcaatggtggtgctaactacaatgagaagttcaagagaagagccaccatgaccgtagacaaatccacgagcacagtctacatggagctgagcagcctgagatctgaggacacggccgtgtattactgtacaatggggggtaacttcttctttgcttattggggccaaggaaccctggtcaccgtctcgagc

SEQ ID NO: 35 - protein, 109 aa, synthetic construct
DVVMTQSPLSLPVTLGQPASISCRSSQSLVHTDGDTYLNWFQQRPGQSPRRLIYKVSNRFSGVPDRFSGSGSGTDFTLKISRVEAEDVGVYYCSQNTHVYTFGQGTKLE

SEQ ID NO: 36 - protein, 109 aa, synthetic construct
DVVMTQSPLSLPVTLGQPASISCRSSQSLVHTDGDTYLNWYQQRPGQSPRRLIYKVSNRFSGVPDRFSGSGSGTDFTLKISRVEAEDVGVYYCSQNTHVYTFGQGTKLE

SEQ ID NO: 37 - protein, 109 aa, synthetic construct
DVVMTQSPLSLPVTLGQPASISCRSSQSLVHTDGDTYLNWYQQRPGQSPRRLIYKVSNRFSGVPDRFSGSGSGTDFTLKISRVEAEDVGVYFCSQNTHVYTFGQGTKLE

SEQ ID NO: 38 - protein, 117 aa, synthetic construct
EVQLVESGGGLVKPGGSLRLSCAASGFTFNSYGMSWVRQAPGKRLEWVSTISSGGTYTYYSDSVKGRFTISRDNAKNSLYLQMNSLRAEDTAVYYCARHDWDFFDYWGQGTLVTVSS

SEQ ID NO: 39 - protein, 117 aa, synthetic construct
EVQLVESGGGLVKPGGSLRLSCAASGFTFNSYGMSWVRQAPGKRLEWVATISSGGTYTYYSDSVKGRFTISRDNAKNSLYLQMNSLRAEDTAVYYCARHDWDFFDYWGQGTLVTVSS

SEQ ID NO: 40 - DNA, 327 bp, synthetic construct
  source 1..327: mol_type = other DNA
gatgttgtgatgactcagtctccactctccctgcccgtcacccttggacagccggcctccatctcctgcagatctagtcagagtcttgtacatactgatggagacacctatttaaattggtttcagcagaggccaggccaatctccaaggcgcctaatttataaagtttccaaccgattttctggggtcccagacagattcagcggcagtgggtcaggcactgatttcacactgaaaatcagcagggtggaggctgaggatgttggggtttattactgctctcaaaatacacatgtttacacgtttggccaggggaccaagctggag

SEQ ID NO: 41 - DNA, 327 bp, synthetic construct
  source 1..327: mol_type = other DNA
gatgttgtgatgactcagtctccactctccctgcccgtcacccttggacagccggcctccatctcctgcagatctagtcagagtcttgtacatactgatggagacacctatttaaattggtaccagcagaggccaggccaatctccaaggcgcctaatttataaagtttccaaccgattttctggggtcccagacagattcagcggcagtgggtcaggcactgatttcacactgaaaatcagcagggtggaggctgaggatgttggggtttattactgctctcaaaatacacatgtttacacgtttggccaggggaccaagctggag

SEQ ID NO: 42 - DNA, 327 bp, synthetic construct
  source 1..327: mol_type = other DNA
gatgttgtgatgactcagtctccactctccctgcccgtcacccttggacagccggcctccatctcctgcagatctagtcagagtcttgtacatactgatggagacacctatttaaattggtaccagcagaggccaggccaatctccaaggcgcctaatttataaagtttccaaccgattttctggggtcccagacagattcagcggcagtgggtcaggcactgatttcacactgaaaatcagcagggtggaggctgaggatgttggggtttatttctgctctcaaaatacacatgtttacacgtttggccaggggaccaagctggag

SEQ ID NO: 43 - DNA, 351 bp, synthetic construct
  source 1..351: mol_type = other DNA
gaggtgcagctggtggagtctgggggaggcctggtcaagcctggggggtccctgagactctcctgtgcagcctctggattcaccttcaatagctatggcatgtcttgggtccgccaggctccagggaagaggctggagtgggtctcaaccattagtagtggtggtacttacacatactattcagacagtgtgaaggggcgattcaccatctccagagacaacgccaagaactcactgtatctgcaaatgaacagcctgagagccgaggacacggctgtgtattactgtgcgagacatgattgggacttctttgactactggggccaaggaaccctggtcaccgtctcgagc

SEQ ID NO: 44 - DNA, 351 bp, synthetic construct
  source 1..351: mol_type = other DNA
gaggtgcagctggtggagtctgggggaggcctggtcaagcctggggggtccctgagactctcctgtgcagcctctggattcaccttcaatagctatggcatgtcttgggtccgccaggctccagggaagaggctggagtgggtcgcaaccattagtagtggtggtacttacacatactattcagacagtgtgaaggggcgattcaccatctccagagacaacgccaagaactcactgtatctgcaaatgaacagcctgagagccgaggacacggctgtgtattactgtgcgagacatgattgggacttctttgactactggggccaaggaaccctggtcaccgtctcgagc

SEQ ID NO: 45 - protein, 110 aa, synthetic construct
DVVMTQSPLSLPVTLGQPASISCRSSQTILHSNGNTYLEWYQQRPGQSPRLLIYKVSNRLSGVPDRFSGSGSGTDFTLKISRVEAEDVGVYYCFQGSHDPFTFGPGTKVD

SEQ ID NO: 46 - protein, 110 aa, synthetic construct
DVVMTQSPLSLPVTLGQPASISCRSSQTILHSNGNTYLEWYQQRPGQSLRLLIYKVSNRLSGVPDRFSGSGSGTDFTLKISRVEAEDVGVYYCFQGSHDPFTFGPGTKVD

SEQ ID NO: 47 - protein, 119 aa, synthetic construct
QVQLVQSGSELKKPGASVKVSCKASGYTFTNFAMNWVRQAPGQGLEYMGWINTNTGEPTYVEEFKGRFVFSLDTSVSTAYLQISSLKAEDTAVYYCTREWRRSYYFDYWGQGTLVTVSS

SEQ ID NO: 48 - protein, 119 aa, synthetic construct
QVQLVQSGSELKKPGASVKVSCKASGYTFTNFAMNWVRQAPGQGLRYMGWINTNTGEPTYVEEFKGRFVFSLDTSVSTAYLQISSLKAEDTAVYYCTREWRRSYYFDYWGQGTLVTVSS

SEQ ID NO: 49 - DNA, 330 bp, synthetic construct
  source 1..330: mol_type = other DNA
gatgttgtgatgactcagtctccactctccctgcccgtcacccttggacagccggcctccatctcctgcagatctagtcagaccattttacatagtaatggaaacacctatttagaatggtaccagcagaggccaggccaatctccaaggctcctaatttataaagtttccaaccgattgtctggggtcccagacagattcagcggcagtgggtcaggcactgatttcacactgaaaatcagcagggtggaggctgaggatgttggggtttattactgctttcaaggttcacatgatccattcacgttcggccctgggaccaaagtggat

SEQ ID NO: 50 - DNA, 357 bp, synthetic construct
  source 1..357: mol_type = other DNA
caggtgcagctggtgcaatctgggtctgagttgaagaagcctggggcctcagtgaaggtttcctgcaaggcttctggatacaccttcactaactttgcaatgaactgggtgcgacaggcccctggacaagggcttaggtacatgggatggataaacaccaacactggagagccaacatatgttgaagagttcaagggacggtttgtcttctccttggacacctctgtcagcacggcatatctgcagatcagcagcctaaaggctgaggacactgccgtgtattactgtacaagagagtggaggaggtcatactactttgactactggggccaaggaaccctggtcaccgtctcgagc

SEQ ID NO: 51 - DNA, 357 bp, synthetic construct
  source 1..357: mol_type = other DNA
caggtgcagctggtgcaatctgggtctgagttgaagaagcctggggcctcagtgaaggtttcctgcaaggcttctggatacaccttcactaactttgcaatgaactgggtgcgacaggcccctggacaagggcttgagtacatgggatggataaacaccaacactggagagccaacatatgttgaagagttcaagggacggtttgtcttctccttggacacctctgtcagcacggcatatctgcagatcagcagcctaaaggctgaggacactgccgtgtattactgtacaagagagtggaggaggtcatactactttgactactggggccaaggaaccctggtcaccgtctcgagc

SEQ ID NO: 52 - DNA, 330 bp, synthetic construct
  source 1..330: mol_type = other DNA
gatgttgtgatgactcagtctccactctccctgcccgtcacccttggacagccggcctccatctcctgcagatctagtcagaccattttacatagtaatggaaacacctatttagaatggtaccagcagaggccaggccaatctctaaggctcctaatttataaagtttccaaccgattgtctggggtcccagacagattcagcggcagtgggtcaggcactgatttcacactgaaaatcagcagggtggaggctgaggatgttggggtttattactgctttcaaggttcacatgatccattcacgttcggccctgggaccaaagtggat